SEQUENCE LISTING (76 sequences)

SEQ ID NO: 1 - DNA, 890 bp, Homo sapiens
ggcggaccgaagaacgcaggaagggggccggggggacccgcccccggccggccgcagccatgaactccaacgtggagaacctacccccgcacatcatccgcctggtgtacaaggaggtgacgacactgaccgcagacccacccgatggcatcaaggtctttcccaacgaggaggacctcaccgacctccaggtcaccatcgagggccctgaggggaccccatatgctggaggtctgttccgcatgaaactcctgctggggaaggacttccctgcctccccacccaagggctacttcctgaccaagatcttccacccgaacgtgggcgccaatggcgagatctgcgtcaacgtgctcaagagggactggacggctgagctgggcatccgacacgtactgctgaccatcaagtgcctgctgatccaccctaaccccgagtctgcactcaacgaggaggcgggccgcctgctcttggagaactacgaggagtatgcggctcgggcccgtctgctcacagagatccacgggggcgccggcgggcccagcggcagggccgaagccggtcgggccctggccagtggcactgaagcttcctccaccgaccctggggccccagggggcccgggaggggctgagggtcccatggccaagaagcatgctggcgagcgcgataagaagctggcggccaagaaaaagacggacaagaagcgggcgctgcgggcgctgcggcggctgtagtgggctctcttcctccttccaccgtgaccccaacctctcctgtcccctccctccaactctgtctctaagttatttaaattatggctggggtcggggagggtacagggggcactgggacctggatttgtttttctaaataaagttggaaaagca

SEQ ID NO: 2 - DNA, 1427 bp, Homo sapiens
accggcgtattcccgccctgcttttcgcccgccgttccgtggcgggaactgaggcgactgtggggacatcagtgatcgtaagtctcctgggcccgttattctcagattaggtgacggagctaagacttcgagaccatctcgtcctttttgtatcgcggaaacctgaggaacgagccggcggcggtgacctgcacgagaagccaggctaactgggtgaagtaccatgcaagcatttcttaaaggtacatccatcagtactaaacccccgctgaccaaggatcgaggagtagctgccagtgcgggaagtagcggagagaacaagaaagccaaacccgttccctgggtggaaaaatatcgcccaaaatgtgtggatgaagttgctttccaggaagaagtggttgcagtgctgaaaaaatctttagaaggagcagatcttcctaatctcttgttttacggaccacctggaactggaaaaacatccactattttggcagcagctagagaactctttgggcctgaacttttccgattaagagttcttgagttaaatgcatctgatgaacgtggaatacaagtagttcgagagaaagtgaaaaattttgctcaattaactgtgtcaggaagtcgctcagatgggaagccgtgtccgccttttaagattgtgattctggatgaagcagattctatgacctcagctgctcaggcagctttaagacgtaccatggagaaggagtcgaaaaccacccgattctgtcttatctgtaactatgtcagtcgaataattgaacccctgacctctagatgttcaaaattccgcttcaagcctctgtcagataaaattcaacagcagcgattactagacattgccaagaaggaaaatgtcaaaattagtgatgagggaatagcttatcttgttaaagtgtcagaaggagacttaagaaaagccattacatttcttcaaagcgctactcgattaacaggtggaaaggagatcacagagaaagtgattacagacattgccggggtaataccagctgagaaaattgatggagtatttgctgcctgtcagagtggctcttttgacaaactagaagctgtggtcaaggatttaatagatgagggtcatgcagcaactcagctcgtcaatcaactccatgatgtggttgtagaaaataacttatctgataaacagaagtctattatcacagaaaaacttgccgaagttgacaaatgcctagcagatggtgctgatgaacatttgcaactcatcagcctttgtgcaactgtgatgcagcagttatctcagaattgttaacgtgaatatatctggatggggggttttgtaaataatgaagttgtaataaaaataaaatgaccaaaagcacctttaaaaaaaaaaaaaaaaaaaaaaaaaaaaaaaaaaaaaa

SEQ ID NO: 3 - DNA, 2120 bp, Homo sapiens
ggagggcagagagaaccgcaacacctggtgccgggtcgggtcgtttccggggctttcagtggccggaagtcgcggcgcctgtactgactctaggaagggctggagttgttttgaatgggcgcccgtaagagaggtgggcaagtacgtgttacagacggccacgccgccctttaggcggtcaaggtggggcgagcagacgttcgcccccctgcagtcggccgggtcactacccaagagcctttggaggcggaagcatggaacggtctgcaaacgttcccgagcgggcctctgcggctctggcgggcgtttcgaacttgggcgccgggcacacgcccagtcccgagagcgctgagggttcccttagcgtcgccctcaccccggccaacccgcggggcgccagagtcctggccctttaaacgccgcgcgtgcctcggcgtcttcgtttcgcgcgcccgcccgcggcgccggcggagcgaacatggacccggctgcgcgggtggtgcgggcgctgtggcctggtgggtgcgccttggcctggaggctgggaggccgcccccagccgctgctacccacgcagagccgggctggcttcgcgggggcggcgggcggcccgagccccgtggctgcagctcgtaaggggagcccgcggctgctgggagctgcggcgctggccctggggggagccctggggctgtaccacacggcgcggtggcacctgcgcgcccaggacctccacgcagagcgctcagccgcgcagctctccctgtccagccgcctgcagctgaccctgtaccagtacaagacgtgtcccttctgcagcaaggtccgagccttcctcgacttccatgccctgccctaccaggtggtggaggtgaaccctgtgcgcagggctgagatcaagttctcctcctacagaaaggtgcccatcctggtggcccaggaaggagaaagctcgcaacaactaaatgactcctctgtcatcatcagcgccctcaagacctacctggtgtcggggcagcccctggaagagatcatcacctactacccagccatgaaggctgtgaacgagcagggcaaggaggtgaccgagttcggcaataagtactggctcatgctcaacgagaaggaggcccagcaagtgtatggtgggaaggaggccaggacggaggagatgaagtggcggcagtgggcggacgactggctggtgcacctgatctcccccaatgtgtaccgcacgcccaccgaggctctggcgtcctttgactacattgtccgcgagggcaagttcggagccgtggagggtgccgtggccaagtacatgggtgcagcggccatgtacctcatcagcaagcgactcaagagcaggcaccgcctccaggacaacgtgcgcgaggacctctatgaggctgctgacaagtgggtggctgctgtgggcaaggaccggcccttcatggggggccagaagccgaatctcgctgatttggcggtgtatggcgtgctgcgtgtgatggaggggctggatgcattcgatgacctgatgcagcacacgcacatccagccctggtacctgcgggtggagagggccatcaccgaggcctccccagcgcactgaatgtccccgcgcagagcagagggaaggcagcggaagacgccagctgccagggcctggggccactgggccagcgcctggcgatactggttgggggcaggatcattctgccccttgtccacgcacccccaccagccctctcgcttctaacacagggcacctgctggggctcagggatgttagggacgagttccagccctgccactgccctggggcgacccctccctgtccctgcctccctgctctgccgcccctcttcctggaccctcagtggctgtcccatggctacatcctgtgggtgggggccctcgacaggacagcaggacggtttgttttcagtggaatcccatccctgggttcccctggttcccactcttcccaagcctcccgggactgggacatgtttgcaataaaggaaaggtttgtggcgcctgtcatggcaggcatctcatggaaaaaaaaaaaaaaaaaaaaaaaaaaaaaaaaaaaaaaaaaaaaaaaaaaaaaaaaa

SEQ ID NO: 4 - DNA, 1766 bp, Homo sapiens
acccgatggctattcgagctctcgatctcggagactggagcgggccattcagaggcgcggggccgggcccggcggacgcttgttgttgtccggccgggggaggcggaggtcgctcgctcgctcgctcggctcgctgactcgccggagcgctctgtggcggtcggcggcaggtcggtcgcgagagcgggctctgtggaagggggcgaggctatgtcgcggtggcagcccggatgggccggcagggccgggagtaacgggacgtcgccgcggagcttcttcccccggatacagtgcggcccgagcggaggccgcggcgccgccctccgatcttgaagagcccgcgctgcgcggagcccgcccccgcctgcgcaccggcaccgacgcggagcgacccagcccagccagacccggcccggcgcggcctgatctaacccagccaggcaggcaatactagcccctctggagcacggagctccttccccaaagacatgaagctattggagaactcgagctttgaagccatcaactcacagctgactgtggagaccggagatgcccacatcattggcaggattgagagctactcatgtaagatggcaggagacgacaaacacatgttcaagcagttctgccaggagggccagccccacgtgctggaggcactttctccaccccagacttcaggactgagccccagcagactcagcaaaagccaaggcggtgaggaggagggccccctcagtgacaagtgcagccgcaagaccctcttctacctgattgccacgctcaatgagtccttcaggcctgactatgacttcagcacagcccgcagccatgagttcagccgggagcccagccttagctgggtggtgaatgcagtcaactgcagtctgttctcagctgtgcgggaggacttcaaggatctgaaaccacagctgtggaacgcggtggacgaggagatctgcctggctgaatgtgacatctacagctataacccagacttggactcagatcccttcggggaggatggtagcctctggtccttcaactacttcttctacaacaagcggctcaagcgaatcgtcttctttagctgccgttccatcagtggctccacctacacaccctcagaggcaggcaacgagctggacatggagctgggggaggaggaggtggaggaagaaagcagaagcaggggcagtggggccgaggagaccagcaccatggaggaggacagggtcccagtgatctgtatttgatgaggaggagccgaggccccagcttcatccagcttcaaccaatgcctggacctgtccacctgagaggcccctggggcctccccagctgctggccagaccctggcgctgccacagtcctggcactgcccaaggccatacctgcctagccctttggctccatcctgtggatgcccactcacccctcagactcctgctgcccatgctgtggccggacttgtcagcagggggcctggtgggaggagcgactgccctgcccaaatgaactgccacagcagggacagctggaccgcagagtttatttttgtatttctactgggcctgcacactccagcccaaagggtctgtggccggaggccccacgagcaggccccagcagtcaccggctctggtcttgggccggccccggtgcccacctgtacccccacctcgcccatttggccgcgtgcactgagtgtcactttgctgcagctcgtttctttccaataaaagtttctgtgacttagaaaa

SEQ ID NO: 5 - DNA, 1584 bp, Homo sapiens
gaacatgacggcgccctgggtggccctcgccctcctctggggatcgctgtggcccggctctgggcgtggggaggctgagacacgggagtgcatctactacaacgccaactgggagctggagcgcaccaaccagagcggcctggagcgctgcgaaggcgagcaggacaagcggctgcactgctacgcctcctgggccaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaagcccaggacatgagtgtctctccagactcagtggatctgaa

SEQ ID NO: 6 - DNA, 1770 bp, Homo sapiens
ccacgcgtccgcacttccagggtcggggagacggaactgcggcgaccatgtatttctggtttatcaaaccgctaacacccagtctaagggcaggttctgtcccattgttatcactatcgaagcagccgatggaggaggggaggtctgagcagagggcggggtgcaggcggaatggccctcgtgccctatgaggagaccacggaatttgggttgcagaaattccacaagcctcttgcaactttttcctttgcaaaccacacgatccagatccggcaggactggagacacctgggagtcgcagcggtggtttgggatgcggccatcgttctttccacatacctggagatgggagctgtggagctcaggggccgctctgccgtggagctgggtgctggcacggggctggtgggcatagtggctgccctgctgggtgctcatgtgactatcacggatcgaaaagtagcattagaatttcttaaatcaaacgttcaagccaacttacctcctcatatccaaactaaaactgttgttaaggagctgacttggggacaaaatttggggagtttttctcctggagaatttgacctgatacttggtgctgatatcatatatttagaagaaacattcacagatcttcttcaaacactggaacatctctgtagcaatcactctgtgattcttttagcatgccgaattcgctatgaacgggataacaacttcttagcaatgctggagaggcaatttattgtgagaaaggttcactacgatcctgaaaaagatgtacatatttacgaagcacagaagagaaaccagaaggaggacttataattggctataatttataagaatgttgtcattgagtgtgtcacttaaggtcttagactgcaaatctaaccatatttaatgaaatgtcttactgtacaaaaagtctaagccaaaggttctcaggggagaaagcacatgtgcagttttaaaacaaagcagtgctttgtcccattgctgtgatttttagtcagactttactcagtctgaaatgcaattaacattaaaggattaagtgtgagatttcgatttatgctatttgtgtatcccatactcctcccttttaataaacagtttccactgatgatatgaagggccggtataaagaagtctttaaatgagtaagctttcttggtaagattaaatcttacaaattatttttaaaaccttgtgatatatacaatgtttagctgagttttctaattttctggatgtaaaacaaaaggtttaacctatacattccttgagctgttagtgctatttaaatcttttgccctgtttaggtcctaaacacttttagttgagtaggatatgagcttttttgggtctcatatcatgctttttgccttaatttcaggtatatatatatataagtaaaggaattaagtaaaaataaaatttcagttactttttaaaagcacctgaaatctggccggatgcggtggctcatgcctgtaatcccaccactttgggaggccgaggcgggcagatcacctgaggtcgggagttcaagaccagcctggccaacatggtgaaaccccatctctactaaaaatacaaaaattagccgggcgtggtgtcgggcgcctgtagtcccagctgctcgggaggctgaggcaggggaatcgcttgaacctgggaggcggaggttgcagtgagctgagattgcgccattgtactccagcctgggggacaggagcgagactccatctcaaaaaaaaaaaaaaa

SEQ ID NO: 7 - DNA, 1257 bp, Homo sapiens
gtcccgacgcctcccgcccccgcagttccttggagagcttggagccgcgcgccggagggaataggaaagcttggttacaacccgggacacccggagcttcaggatggttcgtactaagacatggaccctgaagaagcactttgttggctatcctactaatagtgactttgagttgaagacatctgagctcccacccttaaaaaatggagaggtcctgcttgaagctttgttcctcaccgtggatccctacatgagagtggcagccaaaagattgaaggaaggtgatacaatgatggggcagcaagtggccaaagttgtggaaagtaaaaatgtagccctaccaaaaggaactattgtactggcttctccaggctggacaacgcactccatttctgatgggaaagatctggaaaagctgctgacagagtggccagacacaataccactgtctttggctctggggacagttggcatgccaggcctgactgcctactttggcctacttgaaatctgtggtgtgaagggtggagaaacagtgatggttaatgcagcagctggagctgtgggctcagtcgtggggcagattgcaaagctcaagggctgcaaagttgttggagcagtagggtctgatgaaaaggttgcctaccttcaaaagcttggatttgatgtcgtctttaactacaagacggtagagtctttggaagaaaccttgaagaaagcgtctcctgatggttatgattgttattttgataatgtaggtggagagttttcaaacactgttatcggccagatgaagaaatttggaaggattgccatatgtggagccatctctacatataacagaaccggcccacttcccccaggcccacccccagagattgttatctatcaggagcttcgcatggaagcttttgtcgtctaccgctggcaaggagatgcccgccaaaaagctctgaaggacttgctgaaatgggtcttagagggtaaaatccagtacaaggaatatatcattgaaggatttgaaaacatgccagccgcatttatgggaatgctgaaaggagataatttggggaagacaatagtgaaagcatgaaaaagaggacacatggaatctggaggccatttagatgattagttaatttgtttttcaccatttagcaaaaatgtatactaccttaaatgtcttaagaaatagtactcataatgagtttgagctacttaataaaatacatttaagtggtaaaaaaaaaaaaaaaa

SEQ ID NO: 8 - DNA, 910 bp, Homo sapiens
gtggcttgcggctcgggtggctgagcgcgcggggaaatggccacggggacagaccaggtggtgggactcggcctcgtcgccgttagcctgatcatcttcacctactacaccgcctgggtgattctcttgccattcatcgacagtcagcatgtcatccacaagtatttcctgccccgagcctatgctgtcgccatcccactggctgcaggcctcctgctgctcctgtttgtgggactgttcatctcctatgtgatgctgaagaccaagagagtgaccaagaaggctcagtgaaggtcccgcagggatgaggctgccagccccttctctgcttcccctccagcacagggaccaagtgggggagcctgcagaacctgtccaggcacagtggctcctcaagcctgcctgtcctgcagagtccccatggcatggagcttacacctgactgactggagccccctccccgactcccacttccagaagctaggagggagggatacctggaagactccggtcacctccttcttgctcagggcctaaaagatgctggtcctcccaacctcactctcagactccctgccaccttttcccctgggttctgccgtcttgcctcacttcccctcctgtcacatgctgacgttggacttagcaggttctaaggccacatgtgtgacctctctgacttctcttcctccaccaaggcagctttccttaccctgacacagccccagaccccacaaagccttctggacctggaaagcctggggaaggactgacagaccccaggaccagccctggggctcagggcagccaccccgggccgctgaccgactgacctctcctcacggaggcccagccccaaagccccagggctggcccgtttgggacagctgaccaataaacactgatggtg

SEQ ID NO: 9 - DNA, 1386 bp, Homo sapiens
ggaagccgggattcgccctccggggagcgattggtcctcgggaggggcggggaggtggacgcgggtaccggcggtcgtcgggtcggcagcctttggtcagttggcagcggcaagcgcgctgcggttccggtggcgccatgtcgttctgcagcttcttcgggggcgaggttttccagaatcactttgaacctggcgtttacgtgtgtgccaagtgtggctatgagctgttctccagccgctcgaagtatgcacactcgtctccatggccggcgttcaccgagaccattcacgccgacagcgtggccaagcgtccggagcacaatagatctgaagccttgaaggtgtcctgtggcaagtgtggcaatgggttgggccacgagttcctgaacgacggccccaagccggggcagtcccgattctgaatattcagcagctcgctgaagtttgtccctaaaggcaaagaaacttctgcctcccagggtcactaggcgggcagcccacacccaccccagacggccaccacactgaggccacacgttggccattccaccttggagttggaaccctgggcgtcgagacaggaaggcagggcgcagtggttgaaacatcaggacactcccaaggccccggctctgaacaagaccttttcgtttcttggaaaagagactcatttgctgatggttcatgccttctgctgggacaggcctgggctgtgcagccacactgtcggctgacttagccccctgctcactctaggtgcctccaggaggtgagccctgggtgcagctggtctctgaatgacgttacaccctcaccttcttttcctggccctgtctctggactctcccctgtgaggcccaattccaagacagactctcgtcctcaccgaagcttaggcccacatctcccaggctgcttaggagacagaatggaaacggaggccgcccctgccagccgccctggccctggtcactgcatgatccgctctggtcaaacccttccaggccagccagagtggggatggtctgtgacctgctgggaaggcaggctgatggggcacacccttggcctctcgtccacgaggggagaaacctaaaccctgtttcacaatctgtgcggaagtagcttgcctcacttctgcttaggaaagcggctgttgctccataactctaaccagcacagggctgaggcctgcagtgcacacctgcagggaggcccttcccaaggtgtggtgactgtgccttactgtacatgctcggaggcctggccatataggagggtgggtgatgctgaaatcaccccccatcttaagtaattactttctggagtaatcaggtggaaatccatagacaaatgaaacattcagaaaaaaaaaaaaaaaaaaaa

SEQ ID NO: 10 - DNA, 949 bp, Homo sapiens
gcgctccgggcctggaatccctacgcgtccctttgggtttagcacgatgagctcaatcggcactgggtatgacctgtcagcctctacattctctcctgacggaagagtttttcaagttgaatatgctatgaaggctgtggaaaatagtagtacagctattggaatcagatgcaaagatggtgttgtctttggggtagaaaaattagtcctttctaaactttatgaagaaggttccaacaaaagactttttaatgttgatcggcatgttggaatggcagtagcaggtttgttggcagatgctcgttctttagcagacatagcaagagaagaagcttccaacttcagatctaactttggctacaacattccactaaaacatcttgcagacagagtggccatgtatgtgcatgcatatacactctacagtgctgttagaccttttggctgcagtttcatgttagggtcttacagtgtgaatgacggtgcgcaactctacatgattgacccatcaggtgtttcatacggttattggggctgtgccatcggcaaagccaggcaagctgcaaagacggaaatagagaagcttcagatgaaagaaatgacctgccgtgatatcgttaaagaagttgcaaaaataatttacatagtacatgacgaagttaaggataaagcttttgaactagaactcagctgggttggtgaattaactaatggaagacatgaaattgttccaaaagatataagagaagaagcagagaaatatgctaaggaatctctgaaggaagaagatgaatcagatgatgataatatgtaacatttactccagcatctattgtattttaaatttctactccagtccaatgtaactatttagccctggattatacatactgtccaattttcattaaatttttgtcttataactataaaaaaaaaaaaaaaaaaaaaa

SEQ ID NO: 11 - DNA, 1623 bp, Homo sapiens
gctggtttctgggggtcgtggccttgctcccgctgtgcgggaaaagaatccaggcccttccacgcgcgtgtgggtgcgggggccccgaagtgctcgtggttccccgctaggtctccgctggggcaggaaccggaatcatgggtgggaccaccagcacccgccgggtcaccttcgaggcggacgagaatgagaacatcaccgtggtgaagggcatccggctttcggaaaatgtgattgatcgaatgaaggaatcctctccatctggttcgaagtctcagcggtattctggtgcttatggtgcctcagtttctgatgaagaattgaaaagaagagtagctgaggagctggcattggagcaagccaagaaagaatccgaagatcagaaacgactaaagcaagccaaagagctggaccgagagagggctgctgccaatgagcagttaaccagagccatccttcgggagaggatatgtagcgaggaggaacgcgctaaggcaaagcacctggctaggcagctggaagagaaagaccgagtgctaaagaagcaggatgcattctacaaagaacagctggctagactggaggagaggagctcagagttctacagagtcaccactgaacaatatcagaaagctgctgaagaggtggaagcaaagttcaagcgatatgagtctcatccagtctgtgctgatctgcaggccaaaattcttcagtgttaccgtgagaacacccaccagaccctcaaatgctccgctctggccacccagtatatgcactgtgtcaatcatgccaaacagagcatgcttgagaagggaggataaaaactttcagaatgagcaaaacaccatcaacgttaattccagagatggaacattttttttcctagtgagaaaacaacccatttgaagagaagaccactaatgagaagaccactaaagagagacatcaagaatggattcagcagaatcatttcacgttttgaacagcagcagtttgaagggccaaagccttgatcagggatcagtcattaaaggacactcttgagtattagtaaaccctcttatgatgattaaaagagaagggcagccctctccaccttttggtactttctattcaacttgcactgaccataaaatgtttctcttctgaacaagccccatcatttggtgaacctccaccctaacaaagtaggatggggttgggggctaaattaattggagtggggcgaggagagagccagaaaacatagatccgagggcagcagtgctgggtggagagagccagaaaacagatctggaggcagcagtgctggatggaattgtctaggctgtggcatgttggttttgtctttcttttctcctttgattatgtaagagctatttcattataacttattatggtgattatacaggcaagaagacaaaaaggagagaaaatgtacctcttctactggaataatgtttatgattacaagtgagataaggtatttttatcaatatgaaggcaaccttggctgataaaacctctatagtgaatactcacatctttacttcactcactatcaataataaatatattttctgacaaagactggcaaaaaaaaaaaaaaaaaaaaaaaaaaaa

SEQ ID NO: 12 - DNA, 579 bp, Homo sapiens
acgaggggaaagggcgcagggtttgaaacatggcggacgacgtagaccagcaacaaactaccaacactgtagaggagcccctggatcttatcaggctcagcctagatgagcgaatttatgtgaaaatgagaaatgaccgagagcttcgaggcagattacatgcttatgatcaacatttaaatatgatcttgggagatgtggaagaaactgtgactactatagaaattgatgaagaaacatatgaagagatatataaatcaacgaaacggaatattccaatgctctttgtccggggagatggcgttgtcctggttgcccctccactgagagttggctgaaacaaagaatttgtcctgtatggaaaacgggagactttgtacagtggcctctctaaaagtacaaaacattcataagagaaacctgcatacattttgatattaagaaataattccggggattcttccactcctgaaatgagttgatttgcagataactcacaacttcttaagctaaatggtattttcatttttctcaagctctccaataaatatgaccaccaaaaaaaaaaaaaaaaaaaaa

SEQ ID NO: 13 - DNA, 4493 bp, Homo sapiens
atcgtctgccgctttggtgacttctgacagctctctccatggaaggaggcggcggccgcgatgagccttcagcctgccgggcaggggacgtgaacatggatgaccctaagaaggaagacattcttcttttggccgatgaaaaatttgacttcgatctttcattgtcttcttcgagtgcaaatgaagatgatgaagtcttcttcggaccctttggacataaagaaagatgtattgctgccagcttggaattaaataatccggttcccgaacagcctccgttgcccacatctgagagtccctttgcctggagccctctggccggggagaagttcgtggaggtgtacaaagaagctcacttactggctttacacattgagagcagcagccggaaccaggcagcccaagctgccaagcctgaagaccctcggagccagggcgtggaaagattcatacaggagtcaaaattaaaaataaacctctttgagaaagaaaaggaaatgaagaaaagccccacgtctcttaaaagggagacatactacctgtcagacagccccttgctggggccccctgtgggtgagcctcggctcttggcctcctccccggccctgcccagctctggtgcccaggcccgcctcacccgggcgccggggcctccgcactctgctcatgctttgcccagggaatcatgcactgctcatgctgcaagtcaggcagcgactcagaggaagcccgggaccaaattgctgctgcctcgagcggcctctgttagaggaagaagcatccctggggctgcggagaagcccaagaaagagattccagctagtccttccaggacaaaaatcccagctgagaaggaatcccaccgggatgttctccctgacaaacctgccccgggtgctgtcaatgtgccggccgccggaagccacttgggccagggcaagcgggcgatccctgttccaaacaagttggggctgaagaagaccctgttaaaagcacccggctctaccagcaatctcgcaaggaagtcctcctcggggcctgtttggagcggggcatccagtgcgtgcacatccccagcagtgggcaaagctaaatcaagtgaatttgcaagtattcctgcaaatagctcccggcctctgtcaaacatcagcaagtcaggcagaatgggacccgccatgctgcggccagctctgcctgcaggccctgtgggggcatcctcctggcaggccaagcgggtcgatgtttctgagctggcagcggagcagctcacggcacccccctcagcatcccccacccaaccccagactccggaaggtggcggccagtggctgaactccagttgcgcttggtcagaatcttctcaattgaataagactagaagtatcagacggcgagattcctgtctaaattccaagacaaaggttatgcctactcctacaaatcaatttaaaattcctaagttttctattggtgactccccggacagctcaacaccaaagctttcgcgggcacagcggccgcagtcgtgcacgtcagttggcagggtcactgtccacagcaccccggttagacgctcatctgggccagcaccacaaagcctgctgagcgcacggcgtgtgtcagccttgcccacacccgccagccggcgctgctctggccttccaccgatgacccccaaaacgatgcccagggccgtgggctctcccctgtgtgtgccagctcggagacgttcctctgagccccgcaagaactctgcaatgagaactgaaccaacaagggagagcaacagaaagacagattccaggctggtggatgtgtcccctgacaggggttctcctccttcccgtgtgcctcaggcacttaacttttctccagaggaaagcgattctactttctccaaaagtactgccacagaagtagctcgggaggaagccaagccgggtggagatgcagcccctagtgaggctcttcttgtagatatcaaactggaaccactcgcggtcactccagatgctgcaagccagcccctcattgaccttcctctcatcgacttctgcgataccccagaagcacacgtggctgtaggatctgaaagcaggcctctgatcgacctcatgacaaacactccagacatgaataaaaatgtggccaaaccttcaccggtggtgggacagctcatagacctgagctcccctctgatccagctgagccctgaggctgacaaggagaacgtggattccccactcctcaagttctaagccgaaccaaatcctttgccttgaaagaacagccctaaagtggttttcaaccctcagaaacaagctttaggctggtcgcagtggcttacacttgtaaccctagaacttgggaggctgaggtgggcggattacttgagcccaggagttcgggaccagcctgggaaatatagtgaaactcctgtccctacaaaaaatacaaaaattagccgggtgtggtagtgcatgcctgtagtcccagctacttgggaggctgaagtgggaggatggcctgagctcaaggagatgcaggctgcagtgggctgtgattgtgccactgcactccagcctgggcaccaatgtgagaacctgtcttggaaaaaaaaaaaaagaaacatgttttagtagaagttttatttgaaaaagaaaaataagcataaatatattcccagtgctggagagggtgggctgagggactggggccagcacggaccacccaaggcctctgcttcccgccgccaccctcctcgctgccattctctgggctggaatgtgaagcctcagtcactctaaatgaagaattttcttttgaatgttttgtatgtaaaatagcaagtggctatttttaaagttaagtttgtataaatagttagatattctagatttacattaaattgtaaaataaatggacttattgaagcatatcttgatttttaagcttatcttgattttcaaacatgcatagctatttttatcactctaatcagtaaggctactatctagactcgaatgctttcatacaagtgattttcaaaaattagtcaataaaaattgatgtcagtgcaggcccaggcccgcccccagatacactagtttctaggtctggggccagcctagtaattgttactaggcacacaggtgatgctgactcgatggcctgagacacaccccttgagaagaagctgctctggggagacgagggtatgagtggaaagaggatgggcgaggaggccgggcttacctggacactaaggtgatggacggcgtctgctgggcaggcctcggggaggacccctcagctttgctctcagcaggggcccgacaagctcagtgggcagtggcaggaactgagtgccactggaaagcccattccctttatttagaaaacgagctccaggaagccgctactttgtgtccatttctcttgaggaaacttaccaccttggttgagcggcttcatggcagacagcagcgagccagcggccggactctgtatttcggaccccactccagtgctccctgggtcataccaggatctgcctctgtccacaaagatgagggaaaatgatgactgtgctgtgctctctacttcctgcggttactgcgtgattctaaagctgccactcggaacagcgagtcctctgccgtcgagagcagggaggggtagggtggacaggcgatgccgtggaaagacgtctcggtggaaactgccatggtggaaaggtggcgcgcttctcacggctgagttgctgcgcctgcagacggaagctccccacaggcagagctgcttggatgtgtgagtcatgaagccagagaagccccgctccatgagcagtgactccccaggccctgtgacctccctcctgtcttgcagctcctcctggcaccagtccccagggctctcctgttggtagttcctgcttttcttcttggaaattcctcgtggacctcgagatctttaccctaaaatagttctgttgaatttcaccctggcaatgtaaattgatagcttatcttcacagatgccagacaatggacaactcaccatcagtcctctgctcacctgagacaaatgcatgtctgattgcttcctctgccctattgtttatgtgaaaatgcagattcactgagccagactaaggcatcagtgactgttcctctacctgcctctcacatggagattgtgtattcagtgaaaggctgatcaaagacccaaaggaatgcaacagtttatctcttatctacctatgacctgcgagctgcccaccacccccagttgttgcgcctttccagacagaaccagtgtacatcttacacgtattaattgatgtcctgtgtctccctaatatgtatcaaagcaagctgtgcctcgaccaccttgggcacatatcctcaggacatcatcctgaggctgtgtcactggcatgtccttaaccttggcaaaataaactttctaaattgaaaaaaaaaaaaaaaaaa

SEQ ID NO: 14 - protein, 225 aa, Homo sapiens
MNSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHGGAGGPSGRAEAGRALASGTEASSTDPGAPGGPGGAEGPMAKKHAGERDKKLAAKKKTDKKRALRALRRL

SEQ ID NO: 15 - protein, 363 aa, Homo sapiens
MQAFLKGTSISTKPPLTKDRGVAASAGSSGENKKAKPVPWVEKYRPKCVDEVAFQEEVVAVLKKSLEGADLPNLLFYGPPGTGKTSTILAAARELFGPELFRLRVLELNASDERGIQVVREKVKNFAQLTVSGSRSDGKPCPPFKIVILDEADSMTSAAQAALRRTMEKESKTTRFCLICNYVSRIIEPLTSRCSKFRFKPLSDKIQQQRLLDIAKKENVKISDEGIAYLVKVSEGDLRKAITFLQSATRLTGGKEITEKVITDIAGVIPAEKIDGVFAACQSGSFDKLEAVVKDLIDEGHAATQLVNQLHDVVVENNLSDKQKSIITEKLAEVDKCLADGADEHLQLISLCATVMQQLSQNC

SEQ ID NO: 16 - protein, 377 aa, Homo sapiens
MDPAARVVRALWPGGCALAWRLGGRPQPLLPTQSRAGFAGAAGGPSPVAAARKGSPRLLGAAALALGGALGLYHTARWHLRAQDLHAERSAAQLSLSSRLQLTLYQYKTCPFCSKVRAFLDFHALPYQVVEVNPVRRAEIKFSSYRKVPILVAQEGESSQQLNDSSVIISALKTYLVSGQPLEEIITYYPAMKAVNEQGKEVTEFGNKYWLMLNEKEAQQVYGGKEARTEEMKWRQWADDWLVHLISPNVYRTPTEALASFDYIVREGKFGAVEGAVAKYMGAAAMYLISKRLKSRHRLQDNVREDLYEAADKWVAAVGKDRPFMGGQKPNLADLAVYGVLRVMEGLDAFDDLMQHTHIQPWYLRVERAITEASPAH

SEQ ID NO: 17 - protein, 256 aa, Homo sapiens
MKLLENSSFEAINSQLTVETGDAHIIGRIESYSCKMAGDDKHMFKQFCQEGQPHVLEALSPPQTSGLSPSRLSKSQGGEEEGPLSDKCSRKTLFYLIATLNESFRPDYDFSTARSHEFSREPSLSWVVNAVNCSLFSAVREDFKDLKPQLWNAVDEEICLAECDIYSYNPDLDSDPFGEDGSLWSFNYFFYNKRLKRIVFFSCRSISGSTYTPSEAGNELDMELGEEEVEEESRSRGSGAEETSTMEEDRVPVICI

SEQ ID NO: 18 - protein, 512 aa, Homo sapiens
MTAPWVALALLWGSLWPGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 19 - protein, 218 aa, Homo sapiens
MALVPYEETTEFGLQKFHKPLATFSFANHTIQIRQDWRHLGVAAVVWDAAIVLSTYLEMGAVELRGRSAVELGAGTGLVGIVAALLGAHVTITDRKVALEFLKSNVQANLPPHIQTKTVVKELTWGQNLGSFSPGEFDLILGADIIYLEETFTDLLQTLEHLCSNHSVILLACRIRYERDNNFLAMLERQFIVRKVHYDPEKDVHIYEAQKRNQKEDL

SEQ ID NO: 20 - protein, 329 aa, Homo sapiens
MVRTKTWTLKKHFVGYPTNSDFELKTAELPPLKNGEVLLEALFLTVDPYMRVAAKRLKEGDTMMGQQVAKVVESKNVALPKGTIVLASPGWTTHSISDGKDLEKLLTEWPDTIPLSLALGTVGMPGLTAYFGLLEICGVKGGETVMVNAAAGAVGSVVGQIAKLKGCKVVGAVGSDEKVAYLQKLGFDVVFNYKTVESLEETLKKASPDGYDCYFDNVGGEFSNTVIGQMKKFGRIAICGAISTYNRTGPLPPGPPPEIVIYQELRMEAFVVYRWQGDARQKALKDLLKWVLEGKIQYKEYIIEGFENMPAAFMGMLKGDNLGKTIVKA

SEQ ID NO: 21 - protein, 84 aa, Homo sapiens
MATGTDQVVGLGLVAVSLIIFTYYTAWVILLPFIDSQHVIHKYFLPRAYAVAIPLAAGLLLLLFVGLFISYVMLKTKRVTKKAQ

SEQ ID NO: 22 - protein, 116 aa, Homo sapiens
MSFCSFFGGEVFQNHFEPGVYVCAKCGYELFSSRSKYAHSSPWPAFTETIHADSVAKRPEHNRSEALKVSCGKCGNGLGHEFLNDGPKPGQSRFSIFSSSLKFVPKGKETSASQGH

SEQ ID NO: 23 - protein, 255 aa, Homo sapiens
MSSIGTGYDLSASTFSPDGRVFQVEYAMKAVENSSTAIGIRCKDGVVFGVEKLVLSKLYEEGSNKRLFNVDRHVGMAVAGLLADARSLADIAREEASNFRSNFGYNIPLKHLADRVAMYVHAYTLYSAVRPFGCSFMLGSYSVNDGAQLYMIDPSGVSYGYWGCAIGKARQAAKTEIEKLQMKEMTCRDIVKEVAKIIYIVHDEVKDKAFELELSWVGELTNGRHEIVPKDIREEAEKYAKESLKEEDESDDDNM

SEQ ID NO: 24 - protein, 227 aa, Homo sapiens
MGGTTSTRRVTFEADENENITVVKGIRLSENVIDRMKESSPSGSKSQRYSGAYGASVSDEELKRRVAEELALEQAKKESEDQKRLKQAKELDRERAAANEQLTRAILRERICSEEERAKAKHLARQLEEKDRVLKKQDAFYKEQLARLEERSSEFYRVTTEQYQKAAEEVEAKFKRYESHPVCADLQAKILQCYRENTHQTLKCSALATQYMHCVNHAKQSMLEKGG

SEQ ID NO: 25 - protein, 102 aa, Homo sapiens
MADDVDQQQTTNTVEEPLDLIRLSLDERIYVKMRNDRELRGRLHAYDQHLNMILGDVEETVTTIEIDEETYEEIYKSTKRNIPMLFVRGDGVVLVAPPLRVG

SEQ ID NO: 26 - protein, 720 aa, Homo sapiens
MDDPKKEDILLLADEKFDFDLSLSSSSANEDDEVFFGPFGHKERCIAASLELNNPVPEQPPLPTSESPFAWSPLAGEKFVEVYKEAHLLALHIESSSRNQAAQAAKPEDPRSQGVERFIQESKLKINLFEKEKEMKKSPTSLKRETYYLSDSPLLGPPVGEPRLLASSPALPSSGAQARLTRAPGPPHSAHALPRESCTAHAASQAATQRKPGTKLLLPRAASVRGRSIPGAAEKPKKEIPASPSRTKIPAEKESHRDVLPDKPAPGAVNVPAAGSHLGQGKRAIPVPNKLGLKKTLLKAPGSTSNLARKSSSGPVWSGASSACTSPAVGKAKSSEFASIPANSSRPLSNISKSGRMGPAMLRPALPAGPVGASSWQAKRVDVSELAAEQLTAPPSASPTQPQTPEGGGQWLNSSCAWSESSQLNKTRSIRRRDSCLNSKTKVMPTPTNQFKIPKFSIGDSPDSSTPKLSRAQRPQSCTSVGRVTVHSTPVRRSSGPAPQSLLSARRVSALPTPASRRCSGLPPMTPKTMPRAVGSPLCVPARRRSSEPRKNSAMRTEPTRESNRKTDSRLVDVSPDRGSPPSRVPQALNFSPEESDSTFSKSTATEVAREEAKPGGDAAPSEALLVDIKLEPLAVTPDAASQPLIDLPLIDFCDTPEAHVAVGSESRPLIDLMTNTPDMNKNVAKPSPVVGQLIDLSSPLIQLSPEADKENVDSPLLKF

SEQ ID NO: 27 - RNA, 21 nt, Artificial Sequence
  siRNA-1(44-64) oligonucleotide (sense) for human UBE2S mRNA
ccggccggccgcagccaugaa

SEQ ID NO: 28 - RNA, 21 nt, Artificial Sequence
  siRNA-1(146-166) oligonucleotide (sense) for human UBE2S mRNA
uggcaucaaggucuuucccaa

SEQ ID NO: 29 - RNA, 19 nt, Artificial Sequence
  siRNA-2 oligonucleotide (sense) for human UBE2S mRNA
ggagaacuacgaggaguau

SEQ ID NO: 30 - RNA, 19 nt, Artificial Sequence
  siRNA-3 oligonucleotide (sense) for human UBE2S mRNA
uggcgagcgcgauaagaag

SEQ ID NO: 31 - RNA, 21 nt, Artificial Sequence
  siRNA-1(897-917) oligonucleotide (sense) for human RFC4 mRNA
agggaauagcuuaucuuguua

SEQ ID NO: 32 - RNA, 21 nt, Artificial Sequence
  siRNA-1(189-209) oligonucleotide (sense) for human RFC4 mRNA
cugcacgagaagccaggcuaa

SEQ ID NO: 33 - RNA, 19 nt, Artificial Sequence
  siRNA-2 oligonucleotide (sense) for human RFC4 mRNA
ccgauucugucuuaucugu

SEQ ID NO: 34 - RNA, 19 nt, Artificial Sequence
  siRNA-3 oligonucleotide (sense) for human RFC4 mRNA
ggguaauaccagcugagaa

SEQ ID NO: 35 - RNA, 21 nt, Artificial Sequence
  siRNA-1(2003-2023) oligonucleotide (sense) for human PTGES2 mRNA
cugggacauguuugcaauaaa

SEQ ID NO: 36 - RNA, 21 nt, Artificial Sequence
  siRNA-1(1105-1125) oligonucleotide (sense) for human PTGES2 mRNA
cuggcucaugcucaacgagaa

SEQ ID NO: 37 - RNA, 19 nt, Artificial Sequence
  siRNA-2 oligonucleotide (sense) for human PTGES2 mRNA
aggagaaagcucgcaacaa

SEQ ID NO: 38 - RNA, 19 nt, Artificial Sequence
  siRNA-3 oligonucleotide (sense) for human PTGES2 mRNA
ccgaguucggcaauaagua

SEQ ID NO: 39 - RNA, 21 nt, Artificial Sequence
  siRNA-1(1538-1558) oligonucleotide (sense) for human MAF1 mRNA
cagcuggaccgcagaguuuau

SEQ ID NO: 40 - RNA, 21 nt, Artificial Sequence
  siRNA-1(1031-1051) oligonucleotide (sense) for human MAF1 mRNA
uagccucugguccuucaacua

SEQ ID NO: 41 - RNA, 19 nt, Artificial Sequence
  siRNA-2 oligonucleotide (sense) for human MAF1 mRNA
acgacaaacacauguucaa

SEQ ID NO: 42 - RNA, 19 nt, Artificial Sequence
  siRNA-3 oligonucleotide (sense) for human MAF1 mRNA
gccuuagcuggguggugaa

SEQ ID NO: 43 - RNA, 21 nt, Artificial Sequence
  siRNA-1(626-646) oligonucleotide (sense) for human ACVR2B mRNA
cagcucaugaaugacuuugua

SEQ ID NO: 44 - RNA, 21 nt, Artificial Sequence
  siRNA-1(208-228) oligonucleotide (sense) for human ACVR2B mRNA
caccaucgagcucgugaagaa

SEQ ID NO: 45 - RNA, 19 nt, Artificial Sequence
  siRNA-2 oligonucleotide (sense) for human ACVR2B mRNA
ggcagagugaacgggagau

SEQ ID NO: 46 - RNA, 19 nt, Artificial Sequence
  siRNA-3 oligonucleotide (sense) for human ACVR2B mRNA
ggaacaucaucacauggaa

SEQ ID NO: 47 - RNA, 21 nt, Artificial Sequence
  siRNA-1(754-774) oligonucleotide (sense) for human LOC151194 mRNA
aagguucacuacgauccugaa

SEQ ID NO: 48 - RNA, 21 nt, Artificial Sequence
  siRNA-1(1068-1088) oligonucleotide (sense) for human LOC151194 mRNA
ucgauuuaugcuauuugugua

SEQ ID NO: 49 - RNA, 19 nt, Artificial Sequence
  siRNA-2 oligonucleotide (sense) for human LOC151194 mRNA
ggaauuuggguugcagaaa

SEQ ID NO: 50 - RNA, 19 nt, Artificial Sequence
  siRNA-3 oligonucleotide (sense) for human LOC151194 mRNA
ccagaaggaggacuuauaa

SEQ ID NO: 51 - RNA, 21 nt, Artificial Sequence
  siRNA-1(705-725) oligonucleotide (sense) for human LTB4DH mRNA
cacuguuaucggccagaugaa

SEQ ID NO: 52 - RNA, 21 nt, Artificial Sequence
  siRNA-1(588-608) oligonucleotide (sense) for human LTB4DH mRNA
uggauuugaugucgucuuuaa

SEQ ID NO: 53 - RNA, 19 nt, Artificial Sequence
  siRNA-2 oligonucleotide (sense) for human LTB4DH mRNA
gccaaaagauugaaggaag

SEQ ID NO: 54 - RNA, 19 nt, Artificial Sequence
  siRNA-3 oligonucleotide (sense) for human LTB4DH mRNA
ccugaugguuaugauuguu

SEQ ID NO: 55 - RNA, 21 nt, Artificial Sequence
  siRNA-1(145-165) oligonucleotide (sense) for human DPM2 mRNA
cagcaugucauccacaaguau

SEQ ID NO: 56 - RNA, 21 nt, Artificial Sequence
  siRNA-1(84-104) oligonucleotide (sense) for human DPM2 mRNA
uagccugaucaucuucaccua

SEQ ID NO: 57 - RNA, 19 nt, Artificial Sequence
  siRNA-2 oligonucleotide (sense) for human DPM2 mRNA
gggugauucucuugccauu

SEQ ID NO: 58 - RNA, 19 nt, Artificial Sequence
  siRNA-3 oligonucleotide (sense) for human DPM2 mRNA
uguuugugggacuguucau

SEQ ID NO: 59 - RNA, 21 nt, Artificial Sequence
  siRNA-1(870-890) oligonucleotide (sense) for human SEPX1 mRNA
cagacucucguccucaccgaa

SEQ ID NO: 60 - RNA, 21 nt, Artificial Sequence
  siRNA-1(794-814) oligonucleotide (sense) for human SEPX1 mRNA
cugaaugacguuacacccuca

SEQ ID NO: 61 - RNA, 19 nt, Artificial Sequence
  siRNA-2 oligonucleotide (sense) for human SEPX1 mRNA
gggcgagguuuuccagaau

SEQ ID NO: 62 - RNA, 19 nt, Artificial Sequence
  siRNA-3 oligonucleotide (sense) for human SEPX1 mRNA
ucacuuugaaccuggcguu

SEQ ID NO: 63 - RNA, 21 nt, Artificial Sequence
  siRNA-1(853-873) oligonucleotide (sense) for human PSMA3 mRNA
ccaguccaauguaacuauuua

SEQ ID NO: 64 - RNA, 21 nt, Artificial Sequence
  siRNA-1(686-706) oligonucleotide (sense) for human PSMA3 mRNA
cucagcuggguuggugaauua

SEQ ID NO: 65 - RNA, 19 nt, Artificial Sequence
  siRNA-2 oligonucleotide (sense) for human PSMA3 mRNA
uggcagaugcucguucuuu

SEQ ID NO: 66 - RNA, 19 nt, Artificial Sequence
  siRNA-3 oligonucleotide (sense) for human PSMA3 mRNA
gguguuucauacgguuauu

SEQ ID NO: 67 - RNA, 21 nt, Artificial Sequence
  siRNA-1(546-566) oligonucleotide (sense) for human CHCHD3 mRNA
caggaugcauucuacaaagaa

SEQ ID NO: 68 - RNA, 21 nt, Artificial Sequence
  siRNA-1(1450-1470) oligonucleotide (sense) for human CHCHD3 mRNA
cuggaauaauguuuaugauua

SEQ ID NO: 69 - RNA, 19 nt, Artificial Sequence
  siRNA-2 oligonucleotide (sense) for human CHCHD3 mRNA
cgaagaucagaaacgacua

SEQ ID NO: 70 - RNA, 19 nt, Artificial Sequence
  siRNA-3 oligonucleotide (sense) for human CHCHD3 mRNA
gagaaagaccgagugcuaa

SEQ ID NO: 71 - RNA, 21 nt, Artificial Sequence
  siRNA-1(540-560) oligonucleotide (sense) for human LSM3 mRNA
uccaauaaauaugaccaccaa

SEQ ID NO: 72 - RNA, 21 nt, Artificial Sequence
  siRNA-1(37-57) oligonucleotide (sense) for human LSM3 mRNA
acgacguagaccagcaacaaa

SEQ ID NO: 73 - RNA, 19 nt, Artificial Sequence
  siRNA-2 oligonucleotide (sense) for human LSM3 mRNA
gacguagaccagcaacaaa

SEQ ID NO: 74 - RNA, 19 nt, Artificial Sequence
  siRNA-3 oligonucleotide (sense) for human LSM3 mRNA
acgaaacggaauauuccaa

SEQ ID NO: 75 - RNA, 19 nt, Artificial Sequence
  siRNA-2 oligonucleotide (sense) for human GTSE1 mRNA
gggcaaagcuaaaucaagu

SEQ ID NO: 76 - RNA, 19 nt, Artificial Sequence
  siRNA-3 oligonucleotide (sense) for human GTSE1 mRNA
ugacaaacacuccagacau

(19) United States
(12) Patent Application Publication
(10) Pub. No.: US 2010/0249209 A1
(43) Pub. Date: Sep. 30, 2010

(54) GENE INVOLVED IN IMMORTALIZATION OF HUMAN CANCER CELL AND USE THEREOF

(75) Inventors: Masahiko Nishiyama, Hiroshima (JP); Keiko Hiyama, Hiroshima (JP); Keiji Tanimoto, Hiroshima (JP); Norio Masuko, Saitama (JP)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: KABUSHIKI KAISHA YAKULT HONSHA, Tokyo (JP)

(21) Appl. No.: 12/303,982

(22) PCT Filed: Jun. 8, 2007
(86) PCT No.: PCT/JP2007/061652

§ 371 Date: Dec. 9, 2008

(30) Foreign Application Priority Data
Jun. 9, 2006 (JP) 2006-161350

(51) Int. Cl. A61K 31/7088; C07H 21/04; C12Q 1/68; C07K 16/00; G01N 33/53

(52) U.S. Cl. 514/44 A; 536/23.1; 536/24.3; 536/24.33; 435/6; 530/387.9; 435/7.1; 536/24.5

(57) ABSTRACT

The present invention provides a gene related to cancer cell immortalization (an immortalization determining gene) and a process that is useful for selective cancer treatment targeting a cancer cell having the gene. The present invention determines an immortalized cancer cell using a polynucleotide having a base sequence of at least 15 bases that specifically hybridizes with a continuous base sequence of at least 15 bases within any one of abase sequences represented by SEQ ID Nos. 1 to 13. In the foregoing process, the polynucleotide is used as a primer or probe for detecting an immortalization determining gene that exhibits high expression specifically in an immortalized cancer cell.

### TECHNICAL FIELD

The present invention relates to a gene (immortalization-determining gene) involved in the immortalization of human cancer cells, and a method useful for a selective cancer therapy targeting an immortalized cancer cell having the gene. More specifically, the present invention relates to a method for discriminating an immortalized cancer cell using an immortalization-determining gene as an index, and a reagent for the discrimination. The present invention further relates to a method for screening an active ingredient of a selective anticancer drug (immortalized cancer cell growth inhibitor) targeting immortalized cancer cells, and the anticancer drug (immortalized cancer cell growth inhibitor).

### BACKGROUND ART

Patients are given a poor prognosis for human solid cancer unless radical surgery is performed. This mainly derives from the insufficient effect of the present chemotherapy. Since existing chemotherapy drugs basically influence the nucleic acid biosynthesis, DNA, microtubule, etc., as they inhibit the synthesis process from nucleic acid into protein, they inevitably damage, in addition to cancer cells, healthy cells that are in the process of cell growth. Recent research has identified a number of molecules that are specifically expressed in cancer cells, and inhibitors that target these specific molecules can have a minimal adverse reaction to healthy cells. This advantage allows for a greater dosage of the drug. However, in spite of its active development, such a molecule-targeted drug has the following major defect. The molecule-targeted drug does not necessarily promise a certain effect on a majority of cancer cases; the effect can greatly vary for each case. Moreover, it is usually difficult to precisely estimate the effect before the administration. Such a great variation in a molecule-targeted therapeutic drug in each case can derive from the fact that the drug is directed to the molecules of genes involved in carcinogenesis or metastasis. Carcinogenesis varies among different types of cancer and even within the same cancer; therefore, it is assumed that the effect of the targeted molecules on the cancer growth differs in each case. Conversely, the development of a molecule-targeted therapeutic drug that can exhibit a universal effect on many cancers should become possible by identifying a molecule that has a universal and primary effect on various types of cancers.

In all vertebrates, including humans, each chromosome end contains a simple iteration sequence: “TTAGGG” (about 10 kb in humans and several times greater in mice). This terminal sequence cannot be thoroughly reproduced at every DNA duplication upon cell division, and is gradually shortened (on the basis of about 200 bases in humans). This terminal portion is called a “telomere”. When a telomere is shortened to a certain length (about 5 kb in human noncancerous cells) by repeated cell divisions, the cell division eventually ceases. Cell division again becomes possible by cell canceration, but then, when the curtailment finally comes to the ultimate level (about 2 kb), the cell dies. As disclosed in Non-Patent Document 1, reverse transcriptase “telomerase” serves to extend the shortened telomere to stabilize the telomere length, thereby prolonging the cell division limit. Because the telomerase is not expressed in a healthy cell, the cell division in a healthy cell is limited to several tens of times. However, the telomerase is highly expressed in both a productive cell and an immortalized cancer cell. All cancer cells used to be regarded as immortal; however, reports have revealed that some clinically evident cancers did not exhibit telomerase activities (see Non-Patent Documents 2, 3, etc.). Other reports have also revealed that the compulsory expression of telomerase enzyme component TERT in a noncancerous cell did not produce a cancer cell (see Non-Patent Document 4). Accordingly, at present, canceration and immortalization are recognized as different phenomena.

[Non-Patent Document 1] Harley C B, Mutation Res, 256: 271-82, 1991

[Non-Patent Document 2] Kim N W et al., Science 266: 2011-5, 1994

[Non-Patent Document 3] Hiyama K et al., J Natl Cancer Inst 87: 895-902, 1995

[Non-Patent Document 4] Morales C P et al., Nat Genet. 21: 115-8, 1999

[Non-Patent Document 5] Shay J W, Bacchetti S, Eur J Cancer 33: 787-91, 1997

[Non-Patent Document 6] Hiyama K et al., J Immunol 155: 3711-5, 1995

[Non-Patent Document 7] Hiyama E et al., Int J Oncol 9: 453-8, 1996

[Non-Patent Document 8] Forsyth N R et al., Aging Cell 2: 235-43, 2003

[Non-Patent Document 9] Bodnar A G et al., Exp Cell Res 228: 58-64, 1996

[Non-Patent Document 10] Hiyama K et al., Int J Oncol 27: 87-95, 2005

### DISCLOSURE OF INVENTION

### Technical Problem

The present invention relates to a method for discriminating an immortalized cancer cell, and a reagent used for the discrimination. The present invention further relates to a method for screening an active ingredient for an immortalized cancer cell growth inhibitor that is useful as a selective anticancer drug due to its effect of specifically suppressing immortalized cancer cell growth; and the immortalized cancer cell growth inhibitor.

### Technical Solution

In order to develop a molecule-targeted therapeutic drug that is effective for cancer treatment, especially a drug that is expected to have a universal effect on many types of cancers, the inventors of the present invention focus their attention on another character of cancer, “immortalization”, that is different from “canceration (malignant alteration)”. Specifically, even if cancer cells are found, they will not spread or metastasize to kill the host if their limitless growth can be stopped. The suppression of the limitless growth also increases the possibility of the natural death of cancer cells given that the number of residual cancer cells is reduced by chemotherapy or nonradical surgery to some extent, because it is expected that the cancer cell division term would run out before the next cell growth begins. It has been reported that telomerase expression was seen in 80% or more of several thousands of clinical specimens (see Non-Patent Document 5); telomerase expression was confirmed in various types of cancers examined to date; and telomerase expression was seen in most of the cell strains that derive from human cancers (see Non-Patent Document 2). Based on these reports and the known fact that cancer cells containing telomerase expression will never naturally turn negative again, the immortalization phenomenon seems to exist universally and commonly in many cancer cells, in contrast to the wide variety of carcinogenesis. Accordingly, by targeting the molecule that regulates immortalization, it becomes possible to suppress infinite cancer growth with a universal effect on many types of cancers. Based on this principle, an anti-cancer strategy that directly focuses on telomerase has been tested.

However, the inventors of the present invention had already found that a precursor cell, lymphocyte, etc., of a regenerating organ exhibits telomerase activation even in a normal healthy cell (see Non-Patent Documents 6 and 7). It is also known that cell immortalization is not always induced by compulsive expression of TERT (telomerase enzyme component) in noncancerous cells (see Non-Patent Document 8); and that the healthy precursor cell or lymphocyte that expresses telomerase is not immortalized but has a prolonged life (see Non-Patent Documents 6 and 9). Accordingly, such exhibition of telomerase activation in healthy cells is obviously merely telomerase expression rather than cell immortalization. More specifically, though human cell immortalization substantially requires the activation of a telomerase enzyme that serves to extend the telomere in the chromosome terminus, the immortalization is not induced only by the telomerase activation, but also by the presence of specific molecules for immortalization. The molecules seem to function particularly in cancer cells. In fact, the inventors of the present invention confirmed that a gene that specifically undergoes expression and change in a clone, which has been immortalized by compulsive telomerase expression through the introduction of TERT into healthy cells, completely differs from the reported expression and change seen in cancer cells (see Non-Patent Document 10). This is another fact indicating that the gene involved in the immortalization of healthy cells differs from the gene involved in the immortalization of cancer cells. If the drug is directed to telomerase, it suppresses the division ability of the lymphocyte, blood precursor cell, gut mucosa crypt cell, etc., and thereby tends to damage immunity, hematogenous ability, and gastrointestinal function, which is known to be a fatal harm in cancer treatment. However, if the drug is directed to a immortalization-determining factor that works only in cancer cells, it becomes possible to block the specific infinite proliferation of cancer cells without damaging the healthy trunk/precursor cells having telomerase activities.

Based on this theory, the inventors of the present invention conducted an intensive study to discover genes that are universally involved in the immortalization of human cancer cells. As a result of the research, the inventors of the present invention eventually realized that those genes are not involved in the lifetime extension of cells resulting from telomerase activation in healthy cells, but is specifically involved in the constant proliferation of cancer cells. The inventors then identified thirteen immortalization-determining genes (Table 1) in the following manner. First, the genes that are universally expressed and strengthened in various types of cancers were extracted by microarray assay. Among the obtained genes, thirteen kinds of genes were selected on the basis of their expression in a clinical cancer organ consisting of immortalized cancer cells being stronger than the expression of a cancerous tissue comprised of non-immortalized cancer cells and that undergoing no expression or augmentation in healthy cells expressed by the introduction of telomerase. Then, the inventors confirmed that suppressing the expression of the thirteen genes using siRNA suppresses the proliferation of immortalized cancer cells.

The inventors of the present invention further conducted more detailed research based on the foregoing theory, and eventually completed the present invention. Specifically, the present invention includes the following forms.

Item 1. A marker gene for determining cancer cell immortalization, the marker gene comprising a polynucleotide having a base sequence of at least 15 bases that specifically hybridizes with a continuous base sequence of at least 15 bases within any one of base sequences represented by SEQ ID Nos. 1 to 13.
Item 2. The marker gene according to Item 1, wherein the marker gene is a probe or primer.
Item 3. A method for determining an immortalized cancer cell, comprising the steps of:

(1) bonding the marker gene according to claim 1 or 2 with RNA prepared from a biological sample that is extracted from a test subject and that may contain a cancer cell, or a derivative of the RNA;

(2) measuring an amount of the RNA or the derivative bonded with the marker gene, using the marker gene as an index; and

(3) comparing the amount of RNA or the derivative thereof found in Step (2) (generically referred to as “an RNA amount”, hereinafter) with an amount (generically referred to as “a comparative RNA amount”, hereinafter) of a corresponding RNA or a derivative thereof in a non-immortalized healthy or cancer cell.

Item 4. The method for determining an immortalized cancer cell according to Item 3, further comprising the step of:

(4) determining that the cancer cell of the test subject is immortalized when the RNA amount found in Step (2) is higher than the comparative RNA amount, and determining that the cancer cell of the test subject is not immortalized when the RNA amount found in Step (2) is not higher than the comparative RNA amount.

Item 5. An antibody for recognizing a polypeptide having any one of amino acid sequences represented by SEQ ID Nos. 14 to 26. The antibody is suitable for use in determining an immortalized cancer cell.

Item 6. A method for determining an immortalized cancer cell, comprising the step of:

(1′) bonding a protein-containing fraction containing a polypeptide prepared from a biological sample that is extracted from a test subject and that may contain a cancer cell with the antibody according to Item 5;

(2′) measuring the amount of the polypeptide that is bonded with the antibody, using the antibody as an index; and

(3′) comparing the amount (generically referred to as “a polypeptide amount”, hereinafter) of the polypeptide found in Step (2′) with the amount (generically referred to as “a comparative polypeptide amount”, hereinafter) of a corresponding polypeptide in a non-immortalized healthy or cancer cell.

Item 7. The method for determining an immortalized cancer cell according to Item 6, further comprising the step of:

(4′) determining that the cancer cell of the test subject is immortalized when the polypeptide amount found in Step (2′) is higher than the comparative polypeptide amount, and determining that the cancer cell of the test subject is not immortalized when the polypeptide amount found in Step (2′) is not higher than the comparative polypeptide amount.

Item 8. An immortalized cancer cell determining reagent kit including at least one member selected from the group consisting of the marker gene according to Item 1 and the antibody according to Item 5.
Item 9. A method for screening a substance for suppressing proliferation of immortalized cancer cells, comprising the steps of:

(A) bringing a test material into contact with a cell that can express one of the immortalization-determining genes represented by SEQ ID Nos. 1 to 13;

(B) measuring an expression amount of the immortalization-determining gene in the cell brought into contact with the test material;

(C) comparing the expression amount of the immortalization-determining gene found in Step (B) with an expression amount (generically referred to as “a comparative expression amount”, hereinafter) of an immortalization-determining gene in a cell not in contact with the test material; and

(D) selecting the test material as a candidate substance for suppressing proliferation of immortalized cancer cells when the expression amount found in Step (B) is lower than the comparative expression amount.

Item 10. A method for screening a substance for suppressing proliferation of immortalized cancer cells, comprising the steps of:

(A′) bringing a test material into contact with a polypeptide having one of the amino acid sequences represented by SEQ ID Nos. 14 to 26;

(B′) measuring activity of the polypeptide having one of the amino acid sequences represented by SEQ ID Nos. 14 to 26 brought into contact with the test material;

(C′) determining the degree of the activity of the polypeptide found in Step (B′) by comparing the activity of the polypeptide found in Step (B′) with activity (generically referred to as “a comparative activity”, hereinafter) of a polypeptide not in contact with the test material; and

(D′) selecting the test material as a candidate substance for suppressing proliferation of immortalized cancer cells when the activity of the polypeptide found in Step (B′) is lower than the comparative activity.

Item 11. An immortalized cancer cell growth inhibitor containing, as an active ingredient, a polynucleotide having a base sequence of at least 15 bases that specifically hybridizes with a continuous base sequence of at least 15 bases within any one of base sequences represented by SEQ ID Nos. 1 to 13.
Item 12. The immortalized cancer cell growth inhibitor according to Item 11, wherein the polynucleotide has one of base sequences represented by SEQ ID No. 27 to 76.
Item 13. A method of anticancer treatment comprising the step of administering the immortalized cancer cell growth inhibitor according to Item 11 or 12 to cancer cells of a patient.
Item 14. Use of a polynucleotide having a base sequence of at least 15 bases that specifically hybridizes with a continuous base sequence of at least 15 bases within one of base sequences represented by SEQ ID Nos. 1 to 13, for producing an immortalized cancer cell growth inhibitor.
Item 15. The use according to Item 14, wherein the polynucleotide has one of base sequences represented by SEQ ID Nos. 27 to 76.

### EFFECT OF THE INVENTION

The present invention discovered genes that universally determine the immortalization of many different cancer cells. The genes specifically exhibit greater expression in immortalized cancer cells in a plurality of cancers. The present invention provides a method for discriminating immortalized cancer cells, and a reagent that is used for the discrimination method, which allows for measuring the expression level of the immortalization-determining genes. Referring to the measured level, it becomes possible to distinguish the immortalized cancer cells from finite cells. This differentiation was never possible in conventional general pathological diagnoses. The differentiation thus has great potential as a clinical application that is useful for early diagnosis, new treatment options, prognostification, and the like of various cancers.

Further, with a screening process targeting an immortalization-determining gene of the present invention, an active ingredient of an anticancer drug that specifically suppresses the proliferation of immortalized cancer cells is obtained.

### BEST MODE FOR CARRYING OUT THE INVENTION

### (I) Immortalization-Determining Gene

In the present invention, the term “non-immortalized cancer cells” refers to, among general cancer cells, a finite cell having (1) negative telomerase-activation, (2) a reduced telomere length, and (3) undetectable expression of a telomerase protein. Further, in the present invention, the term “immortalized cancer cell” refers to a cell that lacks at least one of the aforementioned conditions; more specifically, a cell having telomerase activation, a cell having an extended telomere length, and/or a cell detectable expression of a telomerase protein. Such an “immortalized cancer cell” is capable of limitless cell proliferation.

In the present invention, the term “immortalization-determining gene” refers to a gene that determines the immortalization of cancer cells.

The present invention does not particularly limit the type of cancer cell as a target, and includes cells that derive from, for example, lung cancer, breast cancer, esophagus cancer, head and neck cancer, gastric cancer, colon cancer, rectal cancer, liver cancer, gallbladder and bile duct cancer, pancreatic cancer, bladder cancer, prostatic cancer, or uterine cervix carcinoma.

The immortalization-determining genes are, namely, (1) UBE2S gene, (2) RFC4 gene, (3) PTGES2 gene, (4) MAF1 gene, (5) ACVR2B gene, (6) FAM119A gene, (7) LTB4DH gene, (8) DPM2 gene, (9) SEPX1 gene, (10) PSMA3 gene, (11) CHCHD3 gene, (12) LSM3 gene, and (13) GTSE1 gene. Table 1 shows the GenBank accession number, formal gene name, and gene symbol of the foregoing genes, and the SEQ ID No.s indicating the base sequences of the genes. In Table 1, the gene name, gene symbol, etc., are based on NCBI data disclosed on the Internet (http://www.ncbi.nlm.nih.gov/).

TABLE 1

| SEQ ID | GenBank | | Gene |
|---|---|---|---|
| No. | Accession No. | Gene Name | Symbol |
| 1 | NM_014501 | ubiquitin-conjugating enzyme E2S | UBE2S |
| 2 | NM_002916 | replication factor C (activator 1) | RFC4 |
| | | 4, 37 kDa | |
| 3 | NM_025072 | prostaglandin E synthase 2 | PTGES2 |
| 4 | NM_032272 | MAF1 homolog (S. cerevisiae) | MAF1 |
| 5 | NM_001106 | activin A receptor, type IIB | ACVR2B |
| 6 | NM_145280 | family with sequence similarity | FAM119A |
| | | 119, member A | |
| 7 | NM_012212 | leukotriene B4 | LTB4DH |
| | (D49387, | 12-hydroxydehydrogenase | |
| | BQ214856) | | |
| 8 | NM_003863 | dolichyl-phosphate | DPM2 |
| | | mannosyltransferase | |
| | | polypeptide 2, regulatory subunit | |
| 9 | NM_016332 | selenoprotein X, 1 | SEPX1 |
| 10 | NM_002788 | proteasome (prosome, macropain) | PSMA3 |
| | | subunit, alpha type, 3 | |
| 11 | NM_017812 | coiled-coil-helix-coiled-coil-helix | CHCHD3 |
| | | domain containing 3 | |
| 12 | NM_014463 | LSM3 homolog, U6 small nuclear | LSM3 |
| | | RNA associated (S. cerevisiae) | |
| 13 | NM_016426 | G-2 and S-phase expressed 1 | CTSE1 |

As shown in Example 1, the above-listed immortalization-determining genes are all highly expressed in immortalized cancer cells derived from a plurality of cancers, including lung cancer, esophagus cancer, and breast cancer. As shown in Example 2, when the expression of these thirteen genes was suppressed by siRNA in the immortalized cancer cells, in every case, the proliferation of immortalized cancer cells was suppressed. This shows that the thirteen immortalization-determining genes determine the immortalization of cancer cells (immortalization-determining gene).

### (II) Reagent for Judging the Immortalization of Cancer Cells (Marker Gene)

As described above, the immortalization-determining genes ((1) to (13)) having base sequences represented by SEQ ID No.s 1 to 13 are all highly expressed specifically in immortalized cancer cells. Therefore, using the thirteen genes as marker genes, the immortality/mortality of cancer cells can be judged.

The present invention provides an immortalization-determining marker gene (abbreviated as “marker gene” in the present invention) as a tool (reagent) that is suitably used to judge the immortalization of cancer cells. The marker gene is designed as a polynucleotide of at least 15 bases that specifically hybridizes with a continuous base sequence of at least 15 bases among one of the base sequences of the immortalization-determining genes represented by the SEQ ID No.s 1 to 13. The marker gene of the present invention may have an arbitrary form according to the object, and may be any of single-strand DNA, single-strand RNA, double-strand DNA, double-strand RNA, and DNA:RNA hybrid.

The marker gene contains a probe or primer useful for detecting the occurrence of expression of an immortalization-determining gene and/or the level (expression level) in cancer cells to allow for the discrimination of immortalized cancer cells. Further, the probe or primer is also useful as a tool (detection reagent) for detecting the expression variation of an immortalization-determining gene in the screening of a material that suppresses the proliferation of the immortalized cancer cells.

### (II-1) Probe

The immortalization of cancer cells is discriminated by detecting at least one of the above-mentioned immortalization-determining genes: (1) UBE2S gene, (2) RFC4 gene, (3) PTGES2 gene, and (4) MAF1 gene, (5) ACVR2B gene, (6) FAM119A gene, (7) LTB4DH gene, (8) DPM2 gene, (9) SEPX1 gene, (10) PSMA3 gene, (11) CHCHD3 gene, (12) LSM3 gene, and (13) GTSE1 gene, which are highly expressed specifically in the immortalized cancer cells.

The detection of the immortalization-determining genes uses, as probes, polynucleotides, each of which specifically hybridizes with the base sequence of one of the aforementioned immortalization-determining genes. In the present invention, an oligonucleotide having plural base sequences is included in the range of “polynucleotide”.

Each polynucleotide is designed to specifically hybridize with a continuous base sequence of one of the immortalization-determining genes. More specifically, for each of the aforementioned immortalization-determining genes, a corresponding polynucleotide specifically hybridizes with a continuous base of at least 15 bases, preferably 20 bases, and more preferably 30 bases to the total base length of the immortalization-determining gene. Accordingly, each polynucleotide has a base length corresponding to the defined base length.

Throughout the present specification and claims, “specific hybridization” refers to a hybridization that forms only a specific hybrid and does not form a nonspecific hybrid under a stringent hybridization condition. The stringent hybridization condition is determined in the usual manner, for example, based on the melting temperature (Tm) of the nucleic acid at which the hybrid is formed. A typical cleaning condition to ensure the hybridization condition is approximately “1×SSC, 0.1% SDS, 37° C.”, more strictly “0.5×SSC, 0.1% SDS, 42° C.”, and yet more strictly “0.1×SSC, 0.1% SDS, 65° C.”.

The polynucleotide (probe) preferably has a base sequence that is complementary to a continuous base sequence of at least 15 bases of the immortalization-determining gene; however, the probe is not always required to be fully complementary to said continuous base sequence as long as the specific hybridization is possible. The polynucleotide has an identity of at least 70%, preferably not less than 80%, more preferably not less than 90%, yet more preferably not less than 95%, and still more preferably not less than 98%, with respect to either the polynucleotide having a continuous base of at least 15 bases within a base sequence of the immortalization-determining gene or the complementary polynucleotide thereof. The identity of each base sequence can be calculated by way of an identity search, alignment program, BLAST, FASTA, ClustalW, or the like.

Examples of probes suitable for the present invention include oligonucleotides and polynucleotides having a continuous base sequence of 15 bases, preferably 20 bases, and more preferably 30 bases to the total base length of the immortalization-determining gene that hybridizes with at least one polynucleotide (however, when the polynucleotide is RNA, the base “t” in the sequence is replaced with “u”) selected from the group consisting of the following (1) to (13).

(1) A polynucleotide with a continuous base sequence having at least 15 bases from the base sequence (SEQ ID No.1) of the UBE2S gene.
(2) A polynucleotide with a continuous base sequence having at least 15 bases from the base sequence (SEQ ID No.2) of the RFC4 gene.
(3) A polynucleotide with a continuous base sequence having at least 15 bases from the base sequence (SEQ ID No.3) of the PTGES2 gene.
(4) A polynucleotide with a continuous base sequence having at least 15 bases from the base sequence (SEQ ID No.4) of the MAF1 gene.
(5) A polynucleotide with a continuous base sequence having at least 15 bases from the base sequence (SEQ ID No.5) of the ACVR2B gene.
(6) A polynucleotide with a continuous base sequence having at least 15 bases from the base sequence (SEQ ID No.6) of the FAM119A gene.
(7) A polynucleotide with a continuous base sequence having at least 15 bases from the base sequence (SEQ ID No.7) of the LTB4DH gene.
(8) A polynucleotide with a continuous base sequence having at least 15 bases from the base sequence (SEQ ID No.8) of the DPM2 gene.
(9) A polynucleotide with a continuous base sequence having at least 15 bases from the base sequence (SEQ ID No.9) of the SEPX1 gene.
(10) A polynucleotide with a continuous base sequence having at least 15 bases from the base sequence (SEQ ID No.10) of the PSMA3 gene.
(11) A polynucleotide with a continuous base sequence having at least 15 bases from the base sequence (SEQ ID No.11) of the CHCHD3 gene.
(12) A polynucleotide with a continuous base sequence having at least 15 bases from the base sequence (SEQ ID No.12) of the LSM3 gene.
(13) A polynucleotide with a continuous base sequence having at least 15 bases from the base sequence (SEQ ID No.13) of the GTSE1 gene.

These polynucleotides (probes) can be produced by a known method, for example, using a commercially available nucleotide synthesizer, based on the target base sequence of the immortalization-determining gene. The polynucleotides (probes) can also be produced by the PCR method using the target base sequence of the immortalization-determining gene as a template.

More preferably, to simplify detection of the immortalization-determining genes, each probe may be labeled with a radioactive material, a fluorescent material, a chemical luminescent substance or an enzyme (described later in greater detail).

Further, each probe may be immobilized in an arbitrary solid phase before use.

Accordingly, the probes suitable for the present invention include a probe in which an aforementioned polynucleotide is immobilized in a solid phase (for example, an immobilized probe, including a gene chip, a cDNA microarray, an oligo DNA array, or a membrane filter). This probe is suitable for a DNA chip for use in immortalization-determining gene detection, or more specifically, for use in immortalized cancer cell detection.

The solid phase of the immobilized probe (polynucleotide) is not particularly limited as long as it immobilizes the polynucleotide. Examples of solid phases include a glass plate, a nylon membrane, micro beads, a silicon chip, a capillary, and other substrates. The polynucleotide can be immobilized in the solid phase by disposing a synthetic polynucleotide in a solid phase, or by synthesizing a target polynucleotide in a solid phase. The immobilization is a well known technology in the present field, and any suitable method can be used according to the type of immobilized probe. For example, a commercially available spotter (manufactured by Cosmobio Co., Ltd., etc.) is often used to form a DNA microarray. (For example, polynucleotide in situ synthesis using photolithographic technology (AFFYMETRIX CO.), or inkjet technology (ROSETTA INPHARMATICS CO.)

### (II-2) Primer

The present invention provides a polynucleotide as a primer that serves as a marker gene to specifically amplify a base sequence region of each immortalization-determining gene.

The polynucleotide is typically a polynucleotide that has a continuous base sequence of at least 15 bases, preferably 15 to 100 bases, more preferably 15 to 50 bases, yet more preferably 15 to 35 bases, and that specifically hybridizes with a part of at least one polynucleotide (however, when the polynucleotide is RNA, the base “t” in the sequence is replaced with “u”) selected from the group consisting of the following (1) to (13), so as to amplify part or all of the polynucleotide.

(1) A polynucleotide with a continuous base sequence having at least 15 bases from the base sequence (SEQ ID No.1) of the UBE2S gene.
(2) A polynucleotide with a continuous base sequence having at least 15 bases from the base sequence (SEQ ID No.2) of the RFC4 gene.
(3) A polynucleotide with a continuous base sequence having at least 15 bases from the base sequence (SEQ ID No.3) of the PTGES2 gene.
(4) A polynucleotide with a continuous base sequence having at least 15 bases from the base sequence (SEQ ID No.4) of the MAF1 gene.
(5) A polynucleotide with a continuous base sequence having at least 15 bases from the base sequence (SEQ ID No.5) of the ACVR2B gene.
(6) A polynucleotide with a continuous base sequence having at least 15 bases from the base sequence (SEQ ID No.6) of the FAM119A gene.
(7) A polynucleotide with a continuous base sequence having at least 15 bases from the base sequence (SEQ ID No.7) of the LTB4DH gene.
(8) A polynucleotide with a continuous base sequence having at least 15 bases from the base sequence (SEQ ID No.8) of the DPM2 gene.
(9) A polynucleotide with a continuous base sequence having at least 15 bases from the base sequence (SEQ ID No.9) of the SEPX1 gene.
(10) A polynucleotide with a continuous base sequence having at least 15 bases from the base sequence (SEQ ID No.10) of the PSMA3 gene.
(11) A polynucleotide with a continuous base sequence having at least 15 bases from the base sequence (SEQ ID No.11) of the CHCHD3 gene.
(12) A polynucleotide with a continuous base sequence having at least 15 bases from the base sequence (SEQ ID No.12) of the LSM3 gene.
(13) A polynucleotide with a continuous base sequence having at least 15 bases from the base sequence (SEQ ID No.13) of the GTSE1 gene.

As with the probe, each of the defined polynucleotides (primers) preferably has a base sequence complementary to the continuous base sequence of at least 15 bases in one of the immortalization-determining genes; however, as long as a specific hybridization is possible, it is not required to be totally complementary. The polynucleotide preferably has an identity of at least 70%, preferably not less than 80%, more preferably not less than 90%, yet more preferably not less than 95%, and still more preferably not less than 98%, with respect to either the polynucleotide having a continuous base of at least 15 bases within a sequence of the immortalization-determining gene or the complementary polynucleotide thereof.

As with the probes, these polynucleotides (primers) can be produced by a known method, for example, using a commercially available nucleotide synthesizer, based on the target base sequence of the immortalization-determining gene.

### (II-3) Marker

The marker gene (probe or primer) according to the present invention may be a product obtained by adding a marker suitable for detecting an immortalization-determining gene, such as a fluorescent coloring material, an enzyme, a protein, a radioisotope, a chemical luminescent substance, or a biotin, to the aforementioned polynucleotide.

For example, a suitable example of the marker of the present invention made of a fluorescent coloring material, a radioisotope, or a chemical luminescent substance is a general marker capable of labeling a nucleotide to detect or determine the quantity of nucleic acids. Examples of fluorescent coloring materials include, but are not limited to, HEX (4, 7, 2′, 4′, 5′, 7′-hexachloro-6-carboxylfluorescein, green fluorescent coloring material), fluoresceine, NED (product name; Applied Biosystems Inc., yellow fluorescent coloring material), 6-FAM (product name; Applied Biosystems Inc, yellowish green fluorescent coloring material), and rhodamine or its derivative (e.g., tetra methyl rhodamine (TMR)). Fluorescent color labeling on a nucleotide is performed by a suitable method among the known labeling methods (see Nature Biotechnology, 14, 303-308 (1996)). A commercially available fluorescent label kit (for example, oligonucleotide ECL 3′-oligo labeling system produced by Amersham Pharmacia Biotech) can also be used.

The above-described marker gene (an unlabeled or labeled probe or primer) may be used as an immortalization-determining gene detection reagent, in other words, an immortalized cancer cell detection reagent.

### (II-4) Immortalized Cancer Cell Detecting Reagent Kit

The present invention further provides an immortalized cancer cell detecting reagent (marker gene (an unlabeled or labeled probe or primer)) kit. The kit includes at least one of the above mentioned unlabeled or labeled polynucleotides used as probes or primers (the polynucleotide may be immobilized in a solid phase). In addition to the marker gene (probe or primer), the reagent kit of the present invention may include other reagents or tools as required during the actual performance (described later) of the method of the present invention, such as a hybridization reagent, a probe labeling material, a label detecting agent, a buffer solution etc.

### (III) Determination Method for Immortalized Cancer Cells

(III-1) The marker gene (probe or primer) of the present invention enables measurement of the expression amount of the immortalization-determining gene (marker gene) in the cancer cells obtained from a test subject; further, the measured expression amount enables judgment as to whether the cancer cells are “immortalized cancer cells” or “non-immortalized cancer cells”. Existing pathological diagnoses are incapable of determining immortalized cancer cells; however, the immortalized cancer cell determination method of the present invention is capable of detecting the early-stage of incipient immortalized cancer cells even before they develop, thereby allowing for rapid provision of an appropriate cancer treatment for each individual.

In this case, the primer and the probe of the present invention are used as a primer for specifically recognizing and amplifying an RNA resulting from the expression of an immortalization-determining gene or a polynucleotide produced from the RNA (e.g., cDNA), and as a probe for specifically detecting an RNA, or a polynucleotide (e.g., cDNA, DNA amplified from RNA or cDNA) that derives from an RNA, respectively.

More specifically, the marker gene (primer or probe) of the present invention may be used as a primer and/or a probe for specifically detecting an immortalization-determining gene, using a known method for specifically detecting a specific gene, such as the northern blotting method, an RT-PCR method, an in situ hybridization method, or the like. Further, detection and/or quantity determination of the expression amount of the immortalization-determining gene in the cancer cell may be performed using a DNA chip. In this case, the probe of the present invention can be used as a probe for the DNA chip.

For example, the measurement of the expression amount of the immortalization-determining gene by way of the northern blotting method may be performed with the probe of the present invention, or more preferably, by the probe labeled with a radioisotope, a fluorescent material, or a chemical luminescent substance. More specifically, RNA extracted from a cancer cell of a test subject is transferred to a nylon membrane or the like, and the labeled probe is hybridized with the RNA. Then, the amount of the resulting double-strand of the probe and the RNA is measured based on a signal given by the marker of the probe. The detection of the signal may be carried out by an appropriate known tool according to the marker of the probe, such as a radiation detector, or a fluorescence detector. Detection may also be performed with a commercially available northern blotting reagent kit in accordance with the protocol of the kit.

Measurement of the immortalization-determining gene expression amount using the RT-PCR method may also be performed using a primer of the present invention, preferably, a primer labeled with a radioisotope, a fluorescent material, a chemical luminescent substance, or the like. More specifically, a pair of marked primers is hybridized with a template cDNA prepared from RNA deriving from a cancer cell of a test subject, and PCR is performed in the general manner. Then, the amount of the resulting amplified double-strand DNA is measured based on the signal given by the marker of the primer. The detection of the signal may be carried out by an appropriate known tool, such as a radiation detector, or a fluorescence detector. The detection may also be performed with a commercially available RT-PCR reagent kit in accordance with the protocol of the kit.

Measurement of the expression amount of an immortalization-determining gene using a DNA chip assay may also be carried out using a probe of the present invention, preferably, a probe labeled with a radioisotope, a fluorescent material, etc. More specifically, a DNA chip, in which a probe labeled with a radioisotope, a fluorescent material, a chemical luminescent substance, or the like, is fixed to an appropriate carrier. Then, the DNA chip is hybridized with a marked DNA or RNA that is prepared from RNA deriving from a cancer cell of a test subject. Then, the amount of the resulting double-strand of the probe and marked DNA or RNA is measured based on the signal given by the marker of the probe. Detection of the signal may be carried out by an appropriate known tool such as a radiation detector or a fluorescence detector. This method may be performed with a commercially available DNA chip in which DNA corresponding to a probe of the present invention is fixed.

Measurement of the expression amount of immortalization-determining genes includes the following two steps (1) and (2).

(1) a step of bonding a probe or primer of the present invention to RNA prepared from a biological sample that may contain a cancer cell obtained from a test subject, or a derivative of the RNA; and
(2) a step of measuring the amount of the RNA or derivative thereof bonded with the probe or primer, using the probe or primer as an index.

In Step (1), the biological sample can be any sample extracted from a test subject, as long as it has the potential to contain a cancer cell. Examples of the biological sample include body fluid (blood, urine, etc.), tissues, or the extracts thereof, and cultures of the obtained tissues. Extraction of the biological sample from the test object is performed by an appropriate method according to the type of biological sample or type of cancer. Preparation of the RNA from the biological sample is performed by a known method.

Throughout the specification and claims, the term “an RNA derivative” refers to a sample produced by RNA extracted from a biological sample. Examples of an RNA derivative include a complementary polynucleotide (cDNA) prepared by RNA transcription, and DNA amplified from the RNA or cDNA using PCR. The cDNA may be prepared using a known method, and the above-mentioned DNA is prepared by PCR using the primer of the present invention for the immortalization-determining gene. In addition, the probe or primer of the present invention used in Step (1) is preferably labeled with a marker, such as a radioisotope, a fluorescent material, or a chemical luminescent substance.

The amount of the RNA or the RNA derivative found in Step (2) depends on the expression amount of the immortalization-determining gene in the test subject. Accordingly, this amount of RNA or RNA derivative (hereinafter comprehensively referred to as “RNA amount”) found in Step (2) is compared with the amount of the corresponding RNA or RNA derivative (hereinafter comprehensively referred to as “comparative RNA amount”) in non-immortalized healthy or cancer cells to judge the immortalization of the cancer cells of the test subject. More specifically, if the RNA amount of the test subject found in Step (2) is greater than the comparative RNA amount, it is judged that the cancer cells of the test subject are immortalized. In the reverse case, it is judged that the cancer cells of the test subject are not immortalized.

Therefore, in addition to Steps (1) and (2), the immortalized cancer cell determination method of the present invention further includes the following Step (3), and preferably, also Step (4).

(3) a step of comparing the amount of RNA or the RNA derivative (RNA amount) found in Step (2) with the amount of the corresponding RNA or RNA derivative (comparative RNA amount) in a non-immortalized healthy or cancer cell; and
(4) a step of judging that the extracted cancer cell of the test subject is immortalized when the RNA amount in Step (2) is higher than the comparative RNA amount, and judging that the extracted cancer cell of the test subject is not immortalized when the RNA amount in Step (2) is not higher than the comparative RNA amount.

The RNA amount (comparative RNA amount) of the immortalization-determining gene in a non-immortalized healthy or cancer cell is calculated by first measuring the RNA amount of the immortalization-determining gene in a plurality of non-immortalized healthy or cancer cells under the same condition, and then finding a mean or intermediate value of the calculated values.

(III-2) Further, the immortalization of cancer cells can be judged based on the quantity of the expression product of the immortalization-determining gene of the present invention, in other words, based on the production quantity of a polypeptide (immortalization-determining polypeptide, hereinafter) encoded by the aforementioned gene. The production quantity of the immortalization-determining polypeptide can be measured using an antibody that recognizes the polypeptide.

The following are immortalization-determining polypeptides that can be used for the present invention that correspond to the aforementioned immortalization-determining genes (1) to (13).

(14) UBE2S polypeptide: a polypeptide encoded by a UBE2S gene (1) having a base sequence represented by the SEQ ID No.1, namely, a polypeptide having an amino acid sequence represented by the SEQ ID No.14.
(15) RFC4 polypeptide: a polypeptide encoded by a RFC4 gene (2) having a base sequence represented by the SEQ ID No.2, namely, a polypeptide having an amino acid sequence represented by the SEQ ID No.15.
(16) PTGES2 polypeptide: a polypeptide encoded by a PTGES2 gene
(3) having a base sequence represented by the SEQ ID No.3, namely, a polypeptide having an amino acid sequence represented by the SEQ ID No.16.
(17) MAF1 polypeptide: a polypeptide encoded by a MAF1 gene (4) having a base sequence represented by the SEQ ID No.4, namely, a polypeptide having an amino acid sequence represented by the SEQ ID No.17.
(18) ACVR2B polypeptide: a polypeptide encoded by an ACVR2B gene (5) having a base sequence represented by the SEQ ID No.5, namely, a polypeptide having an amino acid sequence represented by the SEQ ID No.18.
(19) FAM119A polypeptide: a polypeptide encoded by a FAM119A gene (6) having a base sequence represented by the SEQ ID No.6, namely, a polypeptide having an amino acid sequence represented by the SEQ ID No.19.
(20) LTB4DH polypeptide: a polypeptide encoded by a LTB4DH gene (7) having a base sequence represented by the SEQ ID No.7, namely, a polypeptide having an amino acid sequence represented by the SEQ ID No.20.
(21) DPM2 polypeptide: a polypeptide encoded by a DPM2 gene (8) having a base sequence represented by the SEQ ID No.8, namely, a polypeptide having an amino acid sequence represented by the SEQ ID No.21.
(22) SEPX1 polypeptide: a polypeptide encoded by a SEPX1 gene (9) having a base sequence represented by the SEQ ID No.9, namely, a polypeptide having an amino acid sequence represented by the SEQ ID No.22.
(23) PSMA3 polypeptide: a polypeptide encoded by a PSMA3 gene (10) having a base sequence represented by the SEQ ID No.10, namely, a polypeptide having an amino acid sequence represented by the SEQ ID No.23.
(24) CHCHD3 polypeptide: a polypeptide encoded by a CHCHD3 gene (11) having a base sequence represented by the SEQ ID No.11, namely, a polypeptide having an amino acid sequence represented by the SEQ ID No.24.
(25) LSM3 polypeptide: a polypeptide encoded by a LSM3 gene (12) having a base sequence represented by the SEQ ID No.12, namely, a polypeptide having an amino acid sequence represented by the SEQ ID No.25.
(26) GTSE1 polypeptide: a polypeptide encoded by a GTSE1 gene (13) having a base sequence represented by the SEQ ID No.13, namely, a polypeptide having an amino acid sequence represented by the SEQ ID No.26.

The (14) UBE2S polypeptide is a publicly known polypeptide, and its production is also known, as disclosed in J. Biol. Chem. 267 (22), 15829-15835 (1992). The (15) RFC4 polypeptide is also publicly known as disclosed in Proc. Natl. Acad. Sci. U.S.A. 89 (12), 5211-5215 (1992). Similarly, the polypeptides are publicly known polypeptides, as the (16) PTGES2 polypeptide is disclosed in Biochem. Biophys. Res. Commun. 291 (4), 884-889 (2002); the (18) ACVR2B polypeptide is disclosed in Mol. Cell. Biol. 16 (3), 1066-1073 (1996); the (20) LTB4DH polypeptide is disclosed in J. Biol. Chem. 271 (5), 2844-2850 (1996); the (21) DPM2 polypeptide is disclosed in EMBO J. 19 (11), 2475-2482 (2000); the (22) SEPX1 polypeptide is disclosed in J. Biol. Chem. 274 (48), 33888-33897 (1999); the (23) PSMA3 polypeptide is disclosed in Biochem. Biophys. Res. Commun. 207 (1), 318-323 (1995); the (25) LSM3 polypeptide is disclosed in EMBO J. 18 (12), 3451-3462 (1999); and the (26) GTSE1 polypeptide is disclosed in Gene 254 (1-2), 229-236 (2000).

The immortalization-determining polypeptides (14) to (26) may also be prepared by a process comprising cloning the corresponding immortalization-determining genes (1) to (13), ligating the genes into a vector plasmid, transforming the genes into host cells, such as E. coli, culturing the transformed cells, and isolating the polypeptides from the culture.

The antibody used in the present invention is not limited as long as it recognizes the immortalization-determining polypeptide, and may be a monoclonal antibody or a polyclonal antibody. The antibody may be an antibody prepared as an antibody against the immortalization-determining polypeptide as an immunizing antigen, or an antigen binding antibody with respect to a polypeptide having at least continuous 8 amino acids, preferably 15 amino acids, and more preferably 20 amino acids out of the amino acid sequences forming the immortalization-determining polypeptide. The polypeptide can be generally synthesized by a publicly known method based on the amino acid sequence of the immortalization-determining polypeptide of the present invention or the base sequence that encodes the amino acid. For example, chemical synthesis using an amino acid synthesizer or gene industrial synthesis can be employed.

The antibody of the present invention can be produced by a general method (see, for example, Current protocols in Molecular Biology, edit. Ausubel et al. (1987), Publish. John Wiley and Sons. Section 11.12-11.13). For example, a polyclonal antibody can be produced by using a polypeptide obtained by expressing a polypeptide in E. coli or a synthetic polypeptide that is given the amino acid sequence portions by a general method, immunizing a test animal with the aforementioned polypeptide, and extracting the antibody from a serum obtained from an immune animal. A monoclonal antibody can also be produced from the polypeptide expressed by E. coli or a synthetic polypeptide that is given the amino acid sequence portions by a general method, immunizing a test animal with the aforementioned polypeptide, fusing the spleen cells and myeloma cells extracted from the test animal to synthesize a hybridoma cell, and extracting the antibody from the hybridoma cell.

These antibodies are also included as a component of the aforementioned cancer cell immortalization determining reagent kit for use in judging the immortalization of cancer cells.

Specifically, judgment of immortalized cancer cells is carried out with the following Step (1′) and Step (2′).

(1′) a step of mixing a protein-containing fraction containing a polypeptide prepared from a biological sample that has the potential to contain cancer cells obtained from a test subject, with an antibody that recognizes the immortalization-determining polypeptide of the present invention; and
(2′) a step of measuring the amount of polypeptide bonded with the antibody, using the antibody as an index.
The amount of polypeptide found in Step (2′) depends on the production quantity of the immortalization-determining polypeptide.

In Step (2′), the biological sample can be any sample extracted from the test subject, as long as it has the potential to contain cancer cells. Examples of biological samples include body fluid (blood, urine, etc.), tissues, or extracts thereof, and cultures of the obtained tissues. Extraction of the biological sample from the test subject is performed by an appropriate method according to the type of biological sample or type of cancer. Preparation of the protein-containing fraction (including polypeptide) from the biological sample is performed by an appropriate combination of known differentiation and purification methods. The immortalization-determining polypeptide can be detected by way of various immunological detection methods, such as western blotting, enzyme immunoassay (EIA), radioisotope immunoassay (RIA), luminescence immunoassay, or the like, using the antibody as a probe.

The following explains Steps (1′) and (2′) for measuring the amount of polypeptide (production quantity of the immortalization-determining polypeptide) by way of western blotting. First, an antibody against the immortalization-determining polypeptide is mixed as a primary antibody with a protein-containing fraction prepared from a biological sample of the test subject to be bonded with the immortalization-determining polypeptide contained in the protein-containing fraction. Next, a secondary antibody labeled with a radioisotope, a fluorescent material, or a chemical luminescent substance is bonded with the primary antibody. Then, the amount of immortalization-determining polypeptide is measured based on the signal that derives from the marker of the secondary antibody. Detection of the signal can be carried out by an appropriate known method using, for example, a radiation detector, or a fluorescence detector.

The immortalization of the cancer cells of the test object can be judged by comparing the obtained amount of polypeptide (production quantity of the immortalization-determining polypeptide) in the test subject in Step (2′) with the amount of comparative immortalization-determining polypeptide (comparative production quantity) in a non-immortalized healthy or cancer cell, and finding the difference level. More specifically, if the obtained amount of polypeptide (production quantity of the immortalization-determining polypeptide) in the test subject in Step (2′) is greater than the amount of comparative polypeptide (comparative production quantity), it is judged that the cancer cells of the test subject are immortalized. In the reverse case, it is judged that the cancer cells of the test subject are not immortalized.

Accordingly, in addition to the aforementioned Steps (1′) and (2′), the immortalized cancer cell determination method of the present invention includes the following Step (3′), and preferably, also Step (4′).

(3′) a step of comparing the amount of polypeptide found in Step (2′) with the amount (comparative polypeptide amount) of the corresponding polypeptide in a non-immortalized healthy or cancer cell; and
(4′) a step of judging that the extracted cancer cell of the test subject is immortalized when the amount of polypeptide found in Step (2′) is higher than the comparative polypeptide amount, and judging that the extracted cancer cell of the test subject is not immortalized when the polypeptide amount is not higher than the comparative polypeptide amount.

The production quantity of the immortalization-determining polypeptide in a non-immortalized healthy or cancer cell is calculated by first measuring the production quantity of the immortalization-determining polypeptide in a plurality of non-immortalized healthy or cancer cells under the same condition, and then finding a mean or intermediate value of the calculated values.

### (IV) A Method for Screening Immortalized Cancer Cell Growth Inhibitor

(IV-1) As shown in the Examples, the immortalization-determining genes ((1) to (13)) according to the present invention exhibit high expression specifically in immortalized cancer cells. Further, by suppressing the expression using siRNA of the immortalization-determining genes, proliferation of the immortalized cancer cells is suppressed. This indicates that the substance that suppresses the expression of the immortalization-determining genes ((1) to (13)) can serve as an immortalized cancer cell growth inhibitor (anticancer drug). Therefore, by using the expression suppression of the immortalization-determining genes ((1) to (13)) as an index, the substance that suppresses proliferation of the immortalized cancer cells can be screened.

Accordingly, the present invention provides a method for screening immortalized cancer cell growth inhibitors using the expression suppression of the immortalization-determining genes ((1) to (13)) as an index.

The present method may include the following Steps (A) to (D).

(A) a step of bringing a test material into contact with a cell that can express one of the immortalization-determining genes (1) to (13);
(B) a step of measuring the expression amount of the immortalization-determining gene in the cell brought into contact with the test material;
(C) a step of comparing the expression amount of the immortalization-determining gene found in Step (B) with the expression amount (comparative expression amount) of the immortalization-determining gene in a cell not in contact with the test material; and
(D) a step of selecting the test material as a substance for inhibiting the proliferation of the immortalized cancer cells when the expression amount found in Step (B) is lower than the comparative expression amount.

The cell used in the present invention can derive from any kind of cell as long as the cell can express at least one of the immortalization-determining genes (1) to (13). Examples of organs from which the cell used may derive include lung, stomach, colon, rectum, liver, gallbladder, bile duct, pancreas, kidney, bladder, prostate gland, womb, marrow, lymph gland and blood. The cell may derive from mammals or birds, preferably mammals, and more preferably humans. The expression of the immortalization-determining gene may be endogenous or exogenous.

The test material is not limited and may be nucleic acid (including polynucleotides), a peptide (including polypeptides), an organic compound, or an inorganic compound. The screening is carried out by bringing a test material or a sample containing the test material (test sample) into contact with a cell that can express the immortalization-determining gene. Examples of the test sample include cell extracts, expression products from a gene library, and extracts of natural vegetable or animal-origin products.

In the screening, the process of bringing the test material into contact with the cell is not limited as long as it does not kill the cell and does not inhibit the expression of the immortalization-determining gene in the cell.

In Steps (B) and (C), the expression amount of the immortalization-determining gene can be found by measuring the amount of mRNA of the immortalization-determining gene or its derivative cDNA or double-strand DNA by northern blotting or RT-PCR, using the aforementioned probe or primer of the present invention. Further, the amount of the immortalization-determining polypeptide as an expression product of the immortalization-determining gene may be used to find the expression amount of the immortalization-determining gene in Steps (B) and (C). The amount of the immortalization-determining polypeptide can be found through various immunological detection methods, such as western blotting, enzyme immunoassay (EIA), radioisotope immunoassay (RIA), luminescence immunoassay, or the like, using an antibody against the immortalization-determining polypeptide.

In Step (C), the substance that inhibits proliferation of the immortalized cancer cells can be screened by comparing the expression amount of the immortalization-determining gene measured in Step (B) with the expression amount of the immortalization-determining gene (comparative expression amount) in a cell not in contact with the test material. More specifically, when the expression amount of the immortalization-determining gene found in Step (B) is lower than the comparative expression amount, the test material can be selected as a substance for inhibiting the proliferation of the immortalized cancer cell.

(IV-2) Activity inhibition of the immortalization-determining polypeptides ((14) to (26)), which are the expression products of the immortalization-determining genes ((1) to (13)) of the present invention also constitutes an index to screen the immortalized cancer cell growth inhibitor. For example, the (14) UBE2S polypeptide is known to have a ubiquitin activator (E2) (J. Biol. Chem. 267 (22), 15829-15835 (1992)); The (16) PTGES2 polypeptide is known to have an activity to convert prostaglandin H2 to prostaglandin E2 (Biochem. Biophys. Res. Commun. 291 (4), 884-889 (2002)); the (18) ACVR2B polypeptide is a transmembrane receptor, and has a Ser/Thr kinase activity in the intercellular domain (Mol. Cell. Biol. 16 (3), 1066-1073 (1996)); the (20) LTB4DH polypeptide has an activity to convert leukotriene B4 to 12-oxo-leukotriene B4 (J. Biol. Chem. 271 (5), 2844-2850 (1996)); and the (22) SEPX1 polypeptide has a methionine sulfoxide reducing activity (Mol. Biol. Cell 15 (3), 1055-1064 (2004)); therefore, inhibitions of these activities of the immortalization-determining polypeptides is particularly useful as indices to screen the immortalized cancer cell growth inhibitor.

More specifically, by carrying out the following Steps (A′) to (D′), a substance for suppressing the proliferation of the immortalized cancer cells can be screened.

(A′) a step of bringing a test material into contact with one of the immortalization-determining polypeptides (14) to (26);
(B′) a step of measuring the activity of the immortalization-determining polypeptide in contact with the test material;
(C′) a step of comparing the activity of the immortalization-determining polypeptide measured in Step (B′) with the activity (comparative activity) of the immortalization-determining polypeptide in an aqueous solution, a cell, or a cell fraction that is not in contact with the test material; and
(D′) a step of selecting the test material as a substance for inhibiting the proliferation of the immortalized cancer cells when the activity found in Step (B′) is lower than the comparative activity.

The screening uses the activity of the immortalization-determining polypeptide as an index, and may be carried out with respect to any object that contains or may contain one of the immortalization-determining polypeptides (14) to (26) (preferably (14), (16), (18), (20) or (22)). An appropriate object is selected according to the desired function (activity) of the immortalization-determining polypeptide. Examples of objects subjected to screening include an aqueous solution containing one of the immortalization-determining polypeptides (14) to (26) (preferably (14), (16), (18), (20) or (22)); a cell that can express one of the immortalization-determining genes (1) to (13) (preferably (1), (3), (5), (7) or (9)); or a cell fraction prepared from the aforementioned cell. The aqueous solution is not particularly limited as long as it contains an immortalization-determining polypeptide. In addition to a general aqueous solution, a cell solution, a nuclear extraction, or a liquid culture supernatant may be used. The cell may be endogenous or exogenous, and may be any cell that can express one of the immortalization-determining genes. The cell fraction designates a fraction that derives from the aforementioned cell, such as a cell membrane fraction, a cytoplasmic fraction, a cell nucleus fraction, or the like.

The same cell and test material as those for the previously described screening can be used for this screening process.

Measurement of the ubiquitin activation capability of the UBE2S polypeptide (14) in Step (B′) can be carried out by a known method. For example, “J. Biol. Chem. 267 (22), 15829-15835 (1992)” discloses a method of performing ubiquitin activation reaction of the UBE2S polypeptide in a system containing ubiquitin, a ubiquitin activating enzyme (E1), and UBE2S polypeptide, and measuring the amount of ubiquitin bonded to the UBE2S polypeptide. Measurement of the ubiquitin amount may be performed by a known method. For example, the reaction is performed using ubiquitin labeled with a radioactive material or enzyme, and the amount of the marker is measured (J. Biol. Chem. 267 (22), 15829-15835 (1992), J. Biol. Chem. 279 (51), 52970-52977 (2004)). Alternatively, measurement can be performed by detecting ubiquitin using an anti-ubiquitin antibody (J. Biol. Chem. 279 (51), 52970-52977 (2004)). The ubiquitin and ubiquitin activating enzyme (E1) may be a marketed commodity, or may be obtained by a known protein synthesis process.

Measurement of the prostaglandin E2 synthesis activity of the PTGES2 polypeptide (16) in Step (B′) may be performed using a known method. For example, as disclosed in “Biochem. Biophys. Res. Commun. 291 (4), 884-889 (2002)”, the production amount of the prostaglandin E2 can be measured by performing a prostaglandin E2 conversion reaction using PTGES2 polypeptide in a system containing PTGES2 polypeptide and prostaglandin H2. The amount of the prostaglandin E2 can be measured using a known method. For example, according to “Proc. Natl. Acad. Sci. U.S.A. 96 (13), 7220-7225 (1999))”, the reaction is performed using prostaglandin H2 labeled with a radioactive material or enzyme, and then the amount of the marker is measured to find the production quantity of the prostaglandin E2. Alternatively, the measurement can be performed by using an anti-prostaglandin E2 antibody (J. Dairy. Sci. 87 (7), 2197-2210 (2004)). The prostaglandin H2 may be a marketed commodity, or may be obtained by a known synthesis process.

Measurement of the Ser/Thr kinase activity of the ACVR2B polypeptide (18) in Step (B′) may be performed using a known method. For example, as disclosed in “Mol. Cell. Biol. 16 (3), 1066-1073 (1996)”, the Ser/Thr kinase activity can be measured by performing a kinase reaction in a system containing ACVR2B polypeptide in the presence of a substrate and ATP, and then measuring the phosphorylation quantity of the substrate. The phosphorylation quantity of the substrate may be measured by a known method. For example, the phosphorylation quantity may be measured by measuring radioactivity using γ³²P-ATP or γ³³P-ATP as a tracer (Mol. Cell. Biol. 16 (3), 1066-1073 (1996)). Alternatively, the phosphorylation quantity may be measured by performing a phosphorylation reaction using a substrate labeled with a fluorescent material or biotin, and measuring the quantity of the phosphorylation marker. Examples of substrates include ACVR2B polypeptide, and specific substrates and peptides.

Measurement of the leukotriene B4 conversion activity of the LTB4DH polypeptide (20) in Step (B′) may be performed using a known method. For example, as disclosed in “J. Biol. Chem. 271 (5), 2844-2850 (1996)”, the leukotriene B4 conversion activity can be measured by performing a leukotriene B4 conversion reaction using LTB4DH polypeptide in a system containing LTB4DH polypeptide and leukotriene B4, and then measuring the production quantity of the 12-oxo-leukotriene B4. The production quantity of the 12-oxo-leukotriene B4 may be measured by a known method, such as HPLC. The leukotriene B4 may be a marketed commodity, or may be obtained by a known chemical synthesis process.

Measurement of the methionine sulfoxide reducing activity of the SEPX1 polypeptide (22) in Step (B′) may be performed using a known method. For example, as disclosed in “Mol. Biol. Cell 15 (3), 1055-1064 (2004)”, the methionine sulfoxide reducing activity can be measured by performing a methionine sulfoxide reducing reaction using SEPX1 polypeptide in a system containing SEPX1 polypeptide and methionine sulfoxide, and then measuring the production quantity of the methionine. The production quantity of the methionine may be measured by a known method, such as HPLC. The methionine sulfoxide may be a marketed commodity, or may be obtained by a known chemical synthesis process.

In Steps (C′) and (D′), the substance that inhibits the proliferation of the immortalized cancer cells can be screened by comparing the measured activity of the immortalization-determining polypeptide in Step (B′) with the comparative activity. More specifically, when the measured activity of the immortalization-determining polypeptide is lower than the comparative activity, the test material is determined to be a substance for inhibiting the proliferation of the immortalized cancer cell.

As explained in the Examples, each of the antisence polynucleotides (siRNA) (SEQ ID No.s 27 to 76) shown in Tables 2 and 3 hybridizes with one of the immortalization-determining genes of the present invention in a cancer cell to suppress the expression of the immortalization-determining gene, thereby suppressing the proliferation of the immortalized cancer cells. Therefore, the antisence polynucleotide (siRNA) can be used as an immortalized cancer cell growth inhibitor. Accordingly, the present invention provides an immortalized cancer cell growth inhibitor containing the antisence polynucleotide as an active ingredient.

The antisence polynucleotide is formed of a polynucleotide of at least 15 bases that specifically hybridizes with a polynucleotide having a continuous base sequence of at least 15 bases within a base sequence of the immortalization-determining genes (1) to (13). The polynucleotide preferably has a base sequence complementary to the continuous base sequence of at least 15 bases in one of the immortalization-determining genes (1) to (13); however, as long as a specific hybridization is possible and thereby the expression of the immortalization-determining gene is suppressed, it is not required to be totally complementary. For example, the base sequence of the polynucleotide may have an identity of 70%, preferably not less than 80%, more preferably not less than 90%, yet more preferably not less than 95%, and still more preferably not less than 98%, with respect to the complementary sequence of the immortalization-determining gene; and the polynucleotide may hybridize with RNA of the immortalization-determining gene to suppress the expression of the immortalization-determining gene. The antisence polynucleotide of the present invention preferably has 15 to 1,000 bases, more preferably 15 to 500 bases, and yet more preferably 16 to 30 bases.

The antisence polynucleotide of the present invention preferably has a base sequence represented by one of SEQ ID No.s 27 to 76, and more preferably a double-strand RNA having one of the base sequences.

The antisence polynucleotide of the present invention may be modified with a known technique to improve stability and cell permeability. For example, a phosphoric residue of each nucleotide may be substituted with a chemically modified phosphoric residue, such as phosphorothioate, methyl phosphonate, or phosphorodithioate, to prevent decomposition by a hydrolase such as nuclease.

The form of the antisence polynucleotide of the present invention is not limited and may be single-strand DNA, single-strand RNA, double-strand DNA, double-strand RNA or DNA:RNA hybrid.

Generally, the antisence polynucleotide of the present invention may be synthesized using a known method, such as chemical synthesis using a synthesizer.

The antisence polynucleotide of the present invention is introduceable into cancer cells of a patient using a virus vector, such as a retrovirus vector, adenovirus vector, or adeno-associated virus vector, or a non-virus vector such as liposome, through an ex vivo process or an in vivo process, thereby suitably serving as an anticancer treatment. Accordingly, the present invention includes an anticancer treatment method comprising the step of administering an immortalized cancer cell growth inhibitor containing the antisence polynucleotide as an active ingredient to a patient.

The immortalized cancer cell growth inhibitor of the present invention contains the aforementioned antisence polynucleotide as an active ingredient, and may also contain a stabilizer, a suspension, a freezing mixture, a buffer solution, a solvent, or the like insofar as the cell proliferation suppression effect of the inhibitor is not impaired. The dosage and administration schedule of the immortalized cancer cell growth inhibitor of the present invention to a cancer patient can be suitably determined by a person having skill in the art according to the type of disease, patient's age, weight, etc. Examples of administration schedules include a 3-week cycle of intravenous administration in which a consecutive 2 to 3-week administration of the antisence polynucleotide is carried out with a dosage of 2 to 10 mg/kg per day; a 1-week cycle of intravenous administration in which consecutive 5-day administration of the antisence polynucleotide is carried out with a dosage of 80 to 120 mg/m² per day; and a 4-week cycle of intravenous administration in which a consecutive 3-week administration of the antisence polynucleotide is carried out with a dosage of 80 to 120 mg/m² per day.

### EXAMPLE

The present invention is more specifically explained below in reference to examples. The present invention is, however, not limited to those examples.

### Example 1

### Identification of Immortalization-Determining Gene

1. Preparation of total RNA from human tissue sample, human cancer cell strain, and human normeoplastic culture cell Using an RNeasy™ Mini kit (Qiagen), total RNAs were extracted from nine kinds of lung cancer cell strains, 21 kinds of esophagus cancer cell strains, nine kinds of digestive system cancers, various other cancer cell strains (gastric cancer, colon cancer, head neck cancer, leukemia), two kinds of normeoplastic esophagus epithelial cells, and two kinds of normal respiratory epithelia, according to the attached protocol. The extracted total RNAs were preserved at −80° C.

Further, total RNAs were also extracted from eleven kinds of breast cancer cell strains, ten kinds of ovary cancer cell strains, ten kinds of pancreas cancer cell strains, one kind of normeoplastic immortalization mammary gland epithelia, one kind of normeoplastic mammary gland epithelia, ten sets of pancreatic cancer tissue and non-cancer pancreas tissue of the same pancreatic cancer patient, the primary focus and three metastasis portions of lung cancer tissue of the same non-small cell lung cancer patient. The obtained total RNAs were preserved. The total RNAs were examined to ensure high quality, with clear 18S and 28S rRNA peaks using a 2100 Bioanalyzer (Agilent Technologies) and RNA LabChip (Agilent Technologies), before they were subjected to a microarray assay.

### 2. Microarray Assay

Using the extracted total RNAs, an exhaustive gene expression assay was carried out by way of a microarray. The microarray used a CodeLink UniSet Human 20K I Bioarray (19881 probe), and comprised (1) a synthesis of cDNA from total RNA, (2) a synthesis of labeled cRNA from the cDNA, (3) a fragmentation process of the labeled cRNA, (4) a hybridization of fragment cRNA and microarray, (5) staining of a microarray, (6) scanning of a microarray, and (7) a gene expression assay.

(1) Synthesis of cDNA from Total RNA

Using a CodeLink Expression Assay Reagent Kit (GE Healthcare Bio Science), a first-strand cDNA was synthesized according to the protocol. More specifically, 1 μg each of the total RNAs obtained in section 1 were adjusted to 10 μl with nuclease-free water, mixed with 1 μl of a bacterial control mRNA diluent solution and 1 μl of T7 oligo (dT) primer, both contained in the kit. The total 12 μl of liquid was heated at 70° C. for 10 minutes, followed by 3 minutes rapid cooling on ice. On the ice, the liquid was mixed with 2 μl of 10× first-strand buffer, 4 μl of 5-mM dNTP Mix, 1 μl of RNase inhibitor, and 1 μl of reverse transcriptase (200 U/μl), all contained in the kit. The total 20 μl of liquid was heated at 42° C. for two hours.

Subsequently, according to the protocol, the RNA in the RNA-DNA hybrid was decomposed, and the RNA strand was substituted with a DNA strand, thereby synthesizing a second-strand cDNA (double-strand cDNA). More specifically, 63 μl of nuclease-free water, 10× second-strand buffer (10 μl), 5-mM dNTP mix (4 μl), DNA polymerase mix (2 μl:10 U/μl), 1 μl of RNase H, all contained in the kit, were added to the 20 μl of first-strand cDNA reaction solution. The total 100 μl of liquid was heated at 16° C. for two hours.

After the reaction, the synthesized double-strand cDNA was purified using a QIA quick PCR purification Kit (QIAGEN) according to the attached protocol. More specifically, 100 μl of the cDNA solution was mixed with 500 μl of buffer PB contained in the kit, and the mixed liquid was placed in a QIA quick spin column set in a 2-ml centrifugal tube and subjected to centrifugation for 50 seconds at 13,000 rpm. After the elution liquid was removed, the QIA quick spin column was set again, 700 μl of buffer PE contained in the kit was added thereto, and 13,000 rpm centrifugation was performed for 1 minute. The elution liquid was removed, the QIA quick spin column was set again, and still another centrifugation was performed at 13,000 rpm for 1 minute so as to completely remove the buffer PE. The QIA quick spin column was set in a new 1.5-ml tube, and 30 μl of nuclease-free water was directly added onto the membrane in the QIA quick spin column, allowed to stand for 1 minute, and followed by 13,000-rpm centrifugation for 1 minute. This process was performed twice (60 μl was used in total). Then, the cDNA solution was concentrated to less than 9.5 μl using a vacuum dryer, and adjusted by adding nuclease-free water to 9.5 μl.

(2) Synthesis of Labeled-cRNA from cDNA

Subsequently, according to the protocol of a CodeLink Expression Assay Reagent Kit (GE Healthcare Bio Science), an in vitro transcription (IVT) reaction was performed in the presence of a biotin-labeled nucleotide to synthesize a cRNA. More specifically, 4.0 μl of a 10×T7 reaction buffer, 4.0 μl of a T7 ATP solution, 4.0 μl of a T7 GTP solution, 4.0 μl of a T7 CTP solution, and 3.0 μl of a T7 UTP solution, all contained in the kit, were mixed. After further adding 7.5 μl of 10-mM biotin-11-UTP (Perkin Elmer Corporation), the total 26.5 μl of liquid was added to the 9.5 μl of cDNA solution prepared in the foregoing process (1). Further, 4.0 μl of a 10×T7 Enzyme Mix contained in the kit was added, and the total 40.0 μl of liquid was mixed well and heated in a vapor phase at 37° C. for 14 hours. After the reaction, the synthesized cRNA was purified using an RNeasy Mini Kit (QIAGEN) according to the attached protocol. More specifically, 60 μl of nuclease-free water was added to the IVT reaction solution (40 μl), and 350 μl of buffer RLT contained in the kit and 250 μl of 100% ethanol was added and mixed well, and the total 700 μl of mixed liquid was placed in an RNeasy mini spin column to be subjected to centrifugation for 15 seconds at 12,000 rpm. The RNeasy mini spin column was set in a new 2-ml centrifugal tube, 500 μl of buffer RPE contained in the kit was added to the column, and another 12,000-rpm centrifugation was performed for 15 seconds. This process was performed twice. After the elution liquid was removed, the RNeasy mini spin column was set in the 2-ml centrifugal tube, and 12,000 rpm centrifugation was performed for 2 minutes to dry the membrane in the column. Thereafter, the RNeasy mini spin column was set in a new 1.5-ml tube, and 50 μl of nuclease-free water was directly added onto the membrane, allowed to stand for 10 minute at room temperature, and followed by 12,000-rpm centrifugation for 1 minute. This process was performed twice (100 μl was used in total).

The sample was mixed well, and dispensed into a 2-μl portion for cRNA quality check using an Agilent 2100 Bioanalyzer and a 2-μl portion diluted 50-fold with sterile distilled water for cRNA quantitative determination; the rest was preserved at −80° C. For the quantitative determination, the absorbency of the 50-fold diluted solution was measured with respect to 260 nm and 280 nm, and the cRNA concentration was determined. The absorbency ratio of 260 nm to 280 nm was confirmed as 1.8 or greater. When the RNA concentration was 0.5 μg/μl or less, the sample was concentrated using a vacuum dryer. The cRNA quality check was performed by electrophoresis using an Agilent 2100 Bioanalyzer and RNA LabChip according to the attached protocol, confirming that the smear peak had at least 500 bases.

(3) Fragmentation of Labeled cRNA

Subsequently, according to the protocol of the CodeLink Expression Assay Reagent Kit (GE Healthcare Bio Science), the cRNA was fragmented into about 100 to 200 bases. More specifically, nuclease-free water was added so that the 10 μg of cRNA became 20 μl; then, 5 μl of 5× fragmentation buffer contained in the kit was added. After heating at 94° C. for 20 minutes, the liquid was rapidly cooled on ice. 78 μl of hybridization buffer A, 130 μl of hybridization buffer B, 27 μl of nuclease-free water, all contained in the kit, were added to the 25 μl of solution containing 10 μg of cRNA fragments, preparing a hybridization solution of 260 μl in total. After vortexing at maximum speed for 5 seconds and spinning-down, the sample was heated at 90° C. for 5 minutes to denaturate the cRNA by heat, followed by cooling on ice.

(4) Hybridization of cRNA Fragment and Microarray

Using a CodeLink Shaker Kit (GE Healthcare Bio Science) and a CodeLink Innova shaker, hybridization was performed according to the protocol. More specifically, a CodeLink uniSet human 20K I bioarray was set in the 12-slide shaker tray contained in the kit. The hybridization solution prepared in the foregoing process (3) was vortexed at maximum speed for 5 seconds and spinned down. The 250 μl of hybridization solution was injected to a right-bottom hole of the sealing chamber of the array, and the hole was covered with a sealing strip (sticker) attached to the array. The shaker tray having an array thereon was placed in the CodeLink Innova shaker, and heated at 37° C. for 18 hours while being rotated at 300 rpm.

### (5) Staining of Microarray

Using a CodeLink Parallel Processing Kit (GE Healthcare Bio Science), the array was stained and washed according to the protocol. More specifically, the sealing chamber was removed from the hybridized array, 13 ml of 0.75×TNT buffer (0.1M Tris-HCl (pH 7.6), 0.15M NaCl, 0.05% Tween 20) was added at room temperature, and the resulting liquid was divided into the slots of a medium reagent reservoir with a bioarray rack contained in the kit. The rack was moved up and down several times to shake off extra hybridization solution. The bioarray rack was then shifted to a large reagent reservoir filled with 240 ml of 0.75×TNT buffer heated to 46° C. The bioarray rack was heated to 46° C. for an hour.

As a staining fluid, for each array, 6.8 μl of Streptavidin-Cy5 liquid dissolved by nuclease-free water to 1 mg/ml was diluted 500-fold using 3393.2 μl of TNB buffer (0.1 M Tris-HCl (pH 7.6), 0.15 M NaCl, 0.5% TSA Blocking Reagent (Perkin Elmer Corporation)) at room temperature. The total 3.4 ml was supplied to fill up the slots of a small reagent reservoir contained in the kit. The bioarray rack holding the array was shifted to the small reagent reservoir, blocked from light with aluminum or the like, and subjected to staining for 30 minutes at room temperature (23° C.±2° C.)

After staining, the bioarray rack was shifted to a large reagent reservoir filled with 240 ml of TNT buffer (room temperature), moved up and down several times and allowed to stand for 5 minutes at room temperature. The rack was then shifted to a large reagent reservoir filled with new TNT buffer (room temperature), moved up and down several times and allowed to stand for 5 minutes at room temperature. This washing process was repeated four times. Finally, the rack was washed with 240 ml of 0.05% Tween 20 solution by being moved up and down for five seconds. This process was repeated twice. The bioarray rack was set in a dried medium reagent reservoir, followed by 3 minutes of centrifugation at room temperature, 600×g.

### (6) Scanning of Microarray

Each of the stained arrays was scanned by an Agilent G2565 scanner (Agilent), to be saved a staining pattern as TIFF image. Each TIFF image was processed by CodeLink expression analysis software, thereby digitizing a signal strength for each gene spot on the array.

### (7) Gene Expression Assay

The signal strength data thus obtained was normalized using GeneSpring GX (Agilent Technologies) microarray gene expression assay software, and the data was then assayed. Specifically, the background signal was subtracted from the spot signal. Values less than 0.01 were equalized to 0.01, and each value was divided by the intermediate value of all spot signals so as to find a standardized relative expression amount of the gene. Genes whose relative expression amount was higher universally in the immortalized cancer cell strains (FIG. 1, lower right) than a healthy cell (FIG. 1, upper left) were discriminated in the following manner. Genes that exhibited enhanced expression universally in various organs appeared to be genes involved in cancer cell immortalization (FIG. 1, large arrow pointing to the right), rather than genes (FIG. 1, downward arrow) involved in canceration of healthy cells (FIG. 1, upper left).

Screened as the first group was any gene in which at least 8 out of 9 kinds of lung cancer cell strains exhibited expression in a quantity of two times or higher than the intermediate value of two normal respiratory epithelia, any gene in which at least 19 out of 21 kinds of esophagus cancer cell strains exhibited expression in a quantity of two times or higher than the intermediate value of two normeoplastic esophagus epithelia, and any gene in which at least 8 out of 9 digestive cancers, or various other cancer cell strains (gastric cancer, colorectal cancer, head and neck cancer, leukemia) exhibited expression in a quantity of two times or higher than the intermediate value of the four kinds of normeoplastic epithelia (two kinds of respiratory epithelia and two kinds of esophagus epithelia). As a result, fifty-one genes were screened (FIG. 2, upper left). As the second group, screened was any gene in which the mean value of the expression levels of the three groups of cancer cell strains of different organs was two times or higher than the mean value of the corresponding epithelia (note that, the esophagus cancer cell strains used for this screening based on the mean value correspond to the seven kinds asseyed first), and in which the expression level of the metastasis lung cancer tissue consisting of immortalization lung cancer cells was two times or higher than the mean value of the primary focus tissue and metastasis tissue consisting of non-immortalization lung cancer cells of the same patient. As a result, 80 genes were extracted (FIG. 2, upper right). Among the two screened groups, seven genes were common.

It is a known fact that cancer cell strains are all immortalized. For each of the immortalization/non-immortalization lung cancer tissues used in the experiments, it was confirmed that the primary focus (FIG. 3, “D2Pr”) and hepatic metastasis focus (FIG. 3, “D5M3”) of the same patient were not immortalized (telomerase activitation: negative; telomere length: reduced; hTERT protein expression: not observed), and that one of the lymph node metastasis focuses (FIG. 3, “D4M2”) substantially consisted only of immortalized cells (high telomerase activation, extended telomere length, hTERT protein expression was observed in almost all cells) and the other metastasis focus (FIG. 3, “D3M1”) consisted of both immortalized cells and non-immortalization cells (low telomerase activation; hTERT protein expression cells were observed among the non-expression cells). These characteristics were confirmed by assaying telomerase activation (Hiyama K et al., J Natl Cancer Inst 87: 895-902, 1995, Case G), telomere length (Hiyama K et al., Oncogene 10: 937-44, 1995, Case 92-D), and hTERT protein in situ expression (Hiyama E et al., Neoplasia 3: 17-26, 2001, FIG. 6A, B).

For further refinement, a gene that exhibits high expression universally in 11 cell strains of breast cancer, 10 cell strains of ovarian cancer, and 10 cell strains of pancreatic cancer was screened. Finally, in total, the thirteen genes: (1) ubiquitin-conjugating enzyme E2S (UBE2S), (2) replication factor C (activator 1) 4, 37 kDa (RFC4), (3) prostaglandin E synthase 2, (PTGES2), (4) MAF1 homolog (S. cerevisiae) (MAF1), (5) activin A receptor, type IIB (ACVR2B), (6) family with sequence similarity 119, member A (FAM119A), (7) leukotriene B4 12-hydroxydehydrogenase (LTB4DH), (8) dolichyl-phosphate mannosyltransferase polypeptide 2, regulatory subunit (DPM2), (9) selenoprotein X, 1 (SEPX1), (10) proteasome (prosome, macropain) subunit, alpha type, 3 (PSMA3), (11) coiled-coil-helix-coiled-coil-helix domain containing 3 (CHCHD3), (12) LSM3 homolog, U6 small nuclear RNA associated (S. cerevisiae) (LSM3), and (13) G-2 and S-phase expressed 1 (GTSE1), were found as genes that exhibit higher expression universally in most lung cancer cell strains, esophagus cancer cell strains, digestive cancer cell strains, breast cancer cell strains, ovarian cancer cell strains, pancreatic cancer cell strains and various other cancer cell strains than mortal normeoplastic cells; exhibit higher expression in a telomerase-positive lung cancer metastasis tissue (immortalized cancer cells) than in a telomerase-negative lung cancer metastasis tissue and primary tissue (non-immortalized cancer cells) of the same patient; and exhibit higher expression in pancreatic cancer cell strains consists only of immortalized cells than in a pancreatic cancer tissue containing both immortalized cells and non-immortalized cells (FIG. 3). The pancreatic cancer tissue used in this experiment showed a higher expression of full length mRNA of the telomerase-coding gene TERT than in the healthy pancreatic tissue of the same patient. However, since the expression was evidently lower compared with the immortalized pancreatic cancer cell strains, it is assumed that the tissue contained non-immortalized cells.

As shown in FIGS. 3 to 15, the foregoing thirteen genes have the following characteristics.

(1) each gene exhibits more enhanced expression in immortalization cells (solid bar in FIGS. 3 to 15, human cancer derived cell strains except for MCF-12A, SV11e, SV11-106) than in normeoplastic mortal cells (open bar in FIGS. 3 to 15);
(2) each gene exhibits more enhanced expression in immortalized cancer cells derived human cancer (solid bar in FIGS. 3 to 15, except for MCF-12A, SV11e, SV11-106) than finite life cancer cells (diagonal lined bar in FIGS. 3 to 15, except for SV12e, SV12-72); and
(3) its expression is less enhanced in normeoplastic telomerase expression life-extension cells obtained by introducing a telomerase-coding gene TERT into healthy lung fibroblasts (diamond-shaped dotted bars in FIGS. 3 to 15; TERT6e, TERT6-85) than in the parent cells (TIG-1). These genes are highly expressed specifically in telomerase-positive immortalized cancer cells. This shows that these genes serve to determine the immortalization of cancer cells (immortalization-determining gene).

Moreover, in the immortalization transformed cells obtained by introducing genes encoding a telomerase-coding gene TERT and SV40 early antigen into healthy lung fibroblasts (FIGS. 3 to 15, SV11e, SV11-106: colony formation and 300 PDL or greater passage capability of passage were confirmed using colony formation assay with soft agar), the expression was enhanced except for MAF1, LTB4DH, than in the parent cells (TIG-1) or normeoplastic life-extension cells (TERT6e, TERT6-85: absence of colony formation and cessation of cell proliferation at 100PDL or less were confirmed using colony formation assay with soft agar). This is presumably because the cells transformed in vitro are not always equal to human cancer cells.

### Example 2

### Suppression of Cancer Cell Proliferation by siRNA (Small Interfering RNA) of Immortalization-Determining Gene

To analyze how the thirteen kinds of immortalization-determining genes identified in Example 1 relate to cancer cell proliferation, siRNAs specified according to the respective gene sequences were designed and created. They were introduced into three kinds of cancer cell strains (HeLa: uterine cervix cancer, KYSE150: esophagus cancer, and HCC50: colon cancer). With these samples, the suppression of mRNA expression of an immortalization-determining gene, and the effect on cell proliferation at 96 hours after introduction were observed.

(1) Designing and Creation of Immortalization-Determining Gene Sequence Specific siRNA (Small Interfering RNA)

In each of the thirteen immortalization-determining genes, two or four kinds of specific siRNA sequences were determined, and Double-strand siRNAs each formed of a sense strand and an antisense strand was purchased from Qiagen and Japan Bio Service. Among the created siRNAs sequences, only the sense strands are shown in Tables 2 and 3. The siRNA-1 of each gene was an equimolar mixture of two kinds.

TABLE 2

| | | | | SEQ |
|---|---|---|---|---|
| Gene | siRNA | | | ID |
| Symbol | Name | Position | Sequence | No. |
| UBE2S | siRNA-1 | 44-64 | CCGGCCGGCCGCAGCCAUGAA | 27 |
| | | 146-166 | UGGCAUCAAGGUCUUUCCCAA | 28 |
| | siRNA-2 | 473-491 | GGAGAACUACGAGGAGUAU | 29 |
| | siRNA-3 | 659-677 | UGGCGAGCGCGAUAAGAAG | 30 |
| RFC4 | siRNA-1 | 897-917 | AGGGAAUAGCUUAUCUUGUUA | 31 |
| | | 189-209 | CUGCACGAGAAGCCAGGCUAA | 32 |
| | siRNA-2 | 745-763 | CCGAUUCUGUCUUAUCUGU | 33 |
| | siRNA-3 | 1023-1041 | GGGUAAUACCAGCUGAGAA | 34 |
| PTGES2 | siRNA-1 | 2003-2023 | CUGGGACAUGUUUGCAAUAAA | 35 |
| | | 1105-1125 | CUGGCUCAUGCUCAACGAGAA | 36 |
| | siRNA-2 | 943-961 | AGGAGAAAGCUCGCAACAA | 37 |
| | siRNA-3 | 1086-1104 | CCGAGUUCGGCAAUAAGUA | 38 |
| MAF1 | siRNA-1 | 1538-1558 | CAGCUGGACCGCAGAGUUUAU | 39 |
| | | 1031-1051 | UAGCCUCUGGUCCWCAACUA | 40 |
| | siRNA-2 | 604-622 | ACGACAAACACAUGUUCAA | 41 |

TABLE 3

| | | | | |
|---|---|---|---|---|
| | siRNA-3 | 856-874 | GCCUUAGCUGGGUGGUGAA | 42 |
| ACVR2B | siRNA-1 | 626-646 | CAGCUCAUGAAUGACUUUGUA | 43 |
| | | 208-228 | CACCAUCGAGCUCGUGAAGAA | 44 |
| | siRNA-2 | 684-702 | GGCAGAGUGAACGGGAGAU | 45 |
| | siRNA-3 | 840-858 | GGAACAUCAUCACAUGGAA | 46 |
| FAM119 | siRNA-1 | 754-774 | AAGGUUCACUACGAUCCUGAA | 47 |
| | | 1068-1088 | UCGAUUUAUGCUAUUUGUGUA | 48 |
| | siRNA-2 | 201-219 | GGAAUUUGGGUUGCAGAAA | 49 |
| | siRNA-3 | 810-828 | CCAGAAGGAGGACUUAUAA | 50 |
| LTB4DH | siRNA-1 | 775-795 | CACUGUUAUCGGCCAGAUGAA | 51 |
| | | 658-678 | UGGAUUUGAUGUCGUCUUUAA | 52 |
| | siRNA-2 | 263-281 | GCCAAAAGAUUGAAGGAAG | 53 |
| | siRNA-3 | 725-743 | CCUGAUGGUUAUGAUUGUU | 54 |
| DPM2 | siRNA-1 | 145-165 | CAGCAUGUCAUCCACAAGUAU | 55 |
| | | 84-104 | UAGCCUGAUCAUCUUCACCUA | 56 |
| | siRNA-2 | 116-134 | GGGUGAUUCUCUUGCCAUU | 57 |
| | siRNA-3 | 224-242 | UGUUUGUGGGACUGUUCAU | 58 |
| SEPX1 | siRNA-1 | 870-890 | CAGACUCUCGUCCUCACCGAA | 59 |
| | | 794-814 | CUGAAUGACGUUACACCCUCA | 60 |
| | siRNA-2 | 161-179 | GGGCGAGGUUUUCCAGAAU | 61 |
| | siRNA-3 | 179-197 | UCACUUUGAACCUGGCGUU | 62 |
| PSMA3 | siRNA-1 | 853-873 | CCAGUCCAAUGUAACUAUUUA | 63 |
| | | 686-706 | CUCAGCUGGGUUGGUGAAUUA | 64 |
| | siRNA-2 | 291-309 | UGGCAGAUGCUCGUUCUUU | 65 |
| | siRNA-3 | 512-530 | GGUGUUUCAUACGGUUAUU | 66 |
| CHCHD3 | siRNA-1 | 546-566 | CAGGAUGCAUUCUACAAAGAA | 67 |
| | | 1450-1470 | CUGGAAUAAUGUUUAUGAUUA | 68 |
| | siRNA-2 | 374-392 | CGAAGAUCAGAAACGACUA | 69 |
| | siRNA-3 | 522-540 | GAGAAAGACCGAGUGCUAA | 70 |
| LSM3 | siRNA-1 | 540-560 | UCCAAUAAAUAUGACCACCAA | 71 |
| | | 37-57 | ACGACGUAGACCAGCAACAAA | 72 |
| | siRNA-2 | 39-57 | GACGUAGACCAGCAACAAA | 73 |
| | siRNA-3 | 261-279 | ACGAAACGGAAUAUUCCAA | 74 |
| GTSE1 | siRNA-2 | 1082-1100 | GGGCAAAGCUAAAUCAAGU | 75 |
| | siRNA-3 | 2116-2134 | UGACAAACACUCCAGACAU | 76 |

### (2) Culture of Human Cancer Cell Strains

The three kinds of cancer cell strains (HeLa: uterine cervix cancer, KYSE150: esophagus cancer, and HCC50: colon cancer) were cultured under the following conditions. HeLa was cultured in a DMEM culture medium (Nacalai Tesque Inc.) containing 10% fetal bovine serum (Sigma Corporation) and gentamycin (Sigma Corporation). KYSE150 and HCC50 were cultured in RPMI 1640 culture media (Nacalai Tesque Inc.) containing 10% Fetal Bovine Serum and gentamycin.

(3) Gene Introduction of Immortalization-Determining Gene Sequence Specific siRNA into Cancer Cell Strain

On the day before the transfection, cancer cells previously cultured were isolated using trypsin. After being suspended in the culture medium detailed in (2) above, HeLa=1.2×10⁴, KYSE150=2×10⁴, and HCC50=1.5×10⁴ were seeded for each well of a 24-well plate. On the day of transfection, the media were replaced with serum-free media Opti-MEM (Invitrogen), and the siRNA shown in Table 2 and 3 were subjected to transfection using Oligofectamine (Invitrogen) or Lipofectamine 2000 (Invitrogen). The concentration of siRNA upon transfection was 40 nM per well. A control (non-silencing) siRNA (Qiagen) was used as a negative control.

(4) Assay of the Suppression of Relative mRNA Expression of Immortalization-Determining Gene

One day after the transfection detailed in (3), total RNA was extracted from each cancer cell strain using RNeasy Mini (Qiagen) according to the instruction book. Using a QuantiTect probe RT-PCR (Qiagen) and an immortalization-determining gene sequence specific TaqMan probe (ABI), quantitative RT-PCR (ABI PRISM 7000 sequence detection system (produced by ABI) was used) was performed under the following conditions.

i) 50° C., 30 minutes,
ii) 95° C., 15 minutes,
iii) 94° C., 15 seconds→60° C., 1 minutes×35 to 45 cycles.

Using β-actin as an internal standard, the expression amount was digitalized based on each amplification curve. As a control, the value of a cell into which NS-sequence siRNA was introduced was used. Measurement of the relative mRNA expression of the immortalization-determining gene was performed twice with the same target gene and the same cancer cell strains under the same conditions.

FIGS. 16 to 28 show the percentages of relative mRNA amounts of immortalization-determining genes in the cells to which each of siRNAs was introduced, when the immortalization-determining gene mRNA percentage in the control is assumed to be 100%. The measurements are shown as mRNA-1 and mRNA-2, respectively, in FIGS. 16 to 28. The suppression of mRNA expression by any one of siRNAs (siRNA-1 to siRNA-3) was observed in all target genes.

### (5) Measurement of the Proliferation of Cancer Cell Strains

The proliferation of cancer cell strains was measured using viable cell count measurement reagent SF (Nacalai Tesque Inc.). At 96 hours after the transfection detailed in (3), 10 μl of WST reagent was added to each well. After 3-hour incubation at 37° C., absorbencies at 450 nm and 595 nm (reference wavelengths) were measured using a plate reader (Wallac ARVO MX1420 Multilabel Counter: produced by Perkin Elmer). According to: absorbency at 450 nm—absorbency at 595 nm—absorbency at BG (only culture medium), the data was digitalized, and a mean value of three wells was found. Then, the value was shown as a percentage when the percentage of NS sequence siRNA introduction cell is assumed to be 100%. Measurement of proliferation of cancer cell strains was performed twice with the same target gene and same cancer cell strains under the same conditions.

The respective cell counts upon siRNA introduction are shown as MTT-1 and MTT-2, respectively, in FIGS. 16 to 28, when the percentage of cell count upon the NS sequence siRNA introduction is assumed to be 100%. In all target genes, the suppression of proliferation by any one of the siRNAs (siRNA-1 to siRNA-3) was seen in three kinds of cancer cells, indicating that those thirteen kinds of immortalization-determining genes can be regarded as being target molecules to attain universal suppression of immortalized cancer cell proliferation.

The discrimination of immortalized cancer cells from mortal cancer cells has significance in functioning as a malum marker, as well as in contributing to remedy development. General cancer diagnosis is based on pathological findings, with which the diagnosis of differentiation, atypism, etc., is also possible. However, general pathologic assays do not allow judgment as to whether the cells are immortalized, in other words, whether the cells have infinite lives. Human telomerase activation has attracted attention to date as an immortalization marker. However, as shown above, telomerase activation has also been seen in some healthy cells; therefore, the new idea that telomerase activation does not always immediately immortalize cells has become knowledge. Further, since the telomerase expression amount is very low, even though immune system staining was established, only a few laboratories have reported their achievements (Hiyama E et al., Neoplasia 3: 17-26, 2001). The use of the marker of the present invention to detect the expression of an immortalization-determining gene allows immortalized cancer cells to be discriminated from finite life cells. Such discrimination was not possible by general conventional pathological diagnosis. The present invention thus has great potential as a clinical application useful for early diagnosis, new treatment options, prognostification, and the like.

### BRIEF DESCRIPTION OF DRAWINGS

FIG. 1 is a schematic view illustrating a mechanism of human cell canceration and immortalization.

FIG. 2 is a schematic view illustrating a target gene extraction process according to the present invention.

FIG. 3 is a graph showing expression levels of UBE2S gene in the various immortalized cancer cells, using an oligo array.

FIG. 4 is a graph showing expression levels of RFC4 gene in the various immortalized cancer cells, using an oligo array.

FIG. 5 is a graph showing expression levels of PTGES2 gene in the various immortalized cancer cells, using an oligo array.

FIG. 6 is a graph showing expression levels of MAF1 gene in the various immortalized cancer cells, using an oligo array.

FIG. 7 is a graph showing expression levels of ACVR2B gene in the various immortalized cancer cells, using an oligo array.

FIG. 8 is a graph showing expression levels of FAM119A gene in the various immortalized cancer cells, using an oligo array.

FIG. 9 is a graph showing expression levels of LTB4DH gene in the various immortalized cancer cells, using an oligo array.

FIG. 10 is a graph showing expression levels of DPM2 gene in the various immortalized cancer cells, using an oligo array.

FIG. 11 is a graph showing expression levels of SEPX1 gene in the various immortalized cancer cells, using an oligo array.

FIG. 12 is a graph showing expression levels of PSMA3 gene in the various immortalized cancer cells, using an oligo array.

FIG. 13 is a graph showing expression levels of CHCHD3 gene in the various immortalized cancer cells, using an oligo array.

FIG. 14 is a graph showing expression levels of LSM3 gene in the various immortalized cancer cells, using an oligo array.

FIG. 15 is a graph showing expression levels of GTSE1 gene in the various immortalized cancer cells, using an oligo array.

FIG. 16 is a graph showing an effect of expression suppression of UBE2S gene and suppression of cell proliferation given by siRNA in cancer cell strains (Example 2).

FIG. 17 is a graph showing an effect of expression suppression of RFC4 gene and suppression of cell proliferation given by siRNA in cancer cell strains (Example 2).

FIG. 18 is a graph showing an effect of expression suppression of PTGES2 gene and suppression of cell proliferation given by siRNA in cancer cell strains (Example 2).

FIG. 19 is a graph showing an effect of expression suppression of MAF1 gene and suppression of cell proliferation given by siRNA in cancer cell strains (Example 2).

FIG. 20 is a graph showing an effect of expression suppression of ACVR2B gene and suppression of cell proliferation given by siRNA in cancer cell strains (Example 2).

FIG. 21 is a graph showing an effect of expression suppression of FAM119A gene and suppression of cell proliferation given by siRNA in cancer cell strains (Example 2).

FIG. 22 is a graph showing an effect of expression suppression of LTB4DH gene and suppression of cell proliferation given by siRNA in cancer cell strains (Example 2).

FIG. 23 is a graph showing an effect of expression suppression of DPM2 gene and suppression of cell proliferation given by siRNA in cancer cell strains (Example 2).

FIG. 24 is a graph showing an effect of expression suppression of SEPX1 gene and suppression of cell proliferation given by siRNA in cancer cell strains (Example 2).

FIG. 25 is a graph showing an effect of expression suppression of PSMA3 gene and suppression of cell proliferation given by siRNA in cancer cell strains (Example 2).

FIG. 26 is a graph showing an effect of expression suppression of CHCHD3 gene and suppression of cell proliferation given by siRNA in cancer cell strains (Example 2).

FIG. 27 is a graph showing an effect of expression suppression of LSM3 gene and suppression of cell proliferation given by siRNA in cancer cell strains (Example 2).

FIG. 28 is a graph showing an effect of expression suppression of GTSE1 gene and suppression of cell proliferation given by siRNA in cancer cell strains (Example 2).

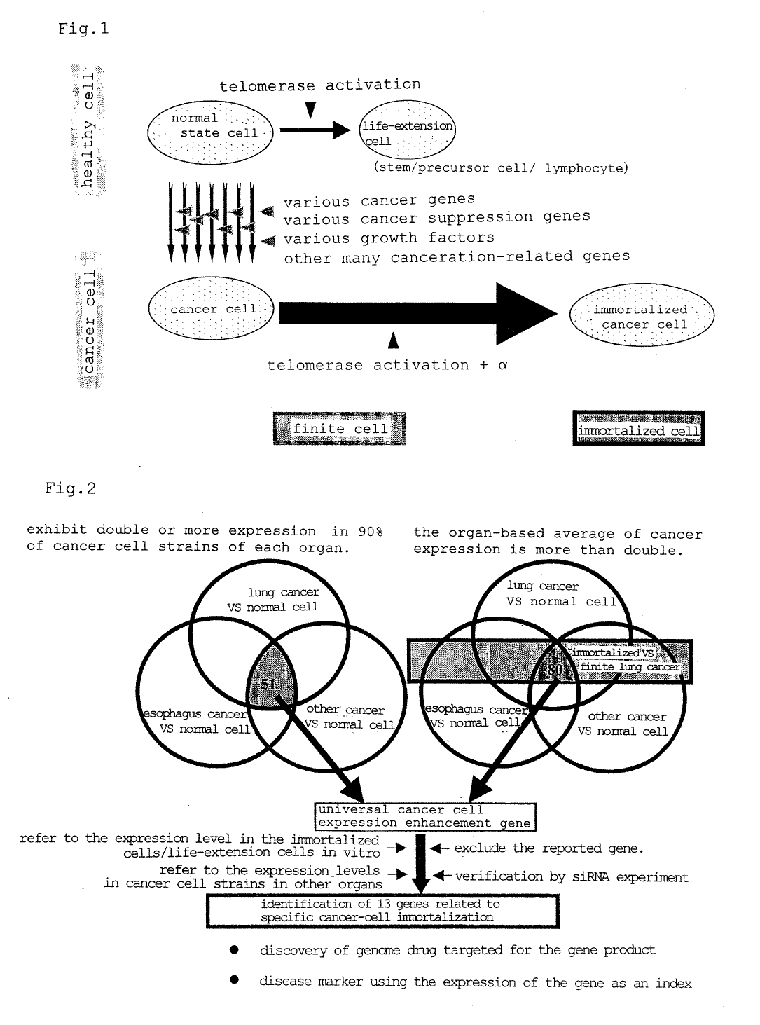

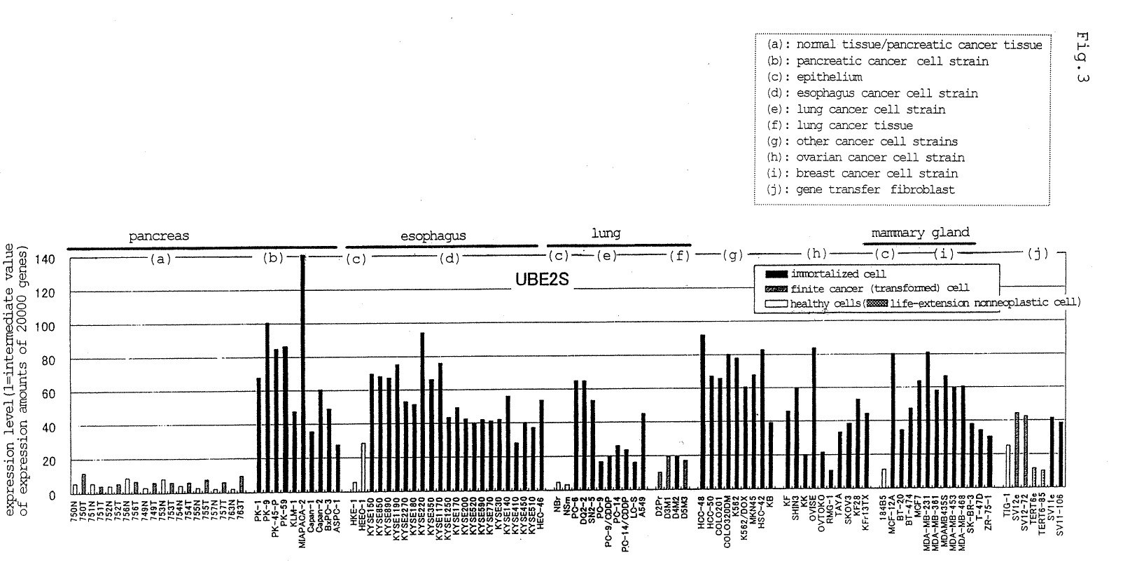

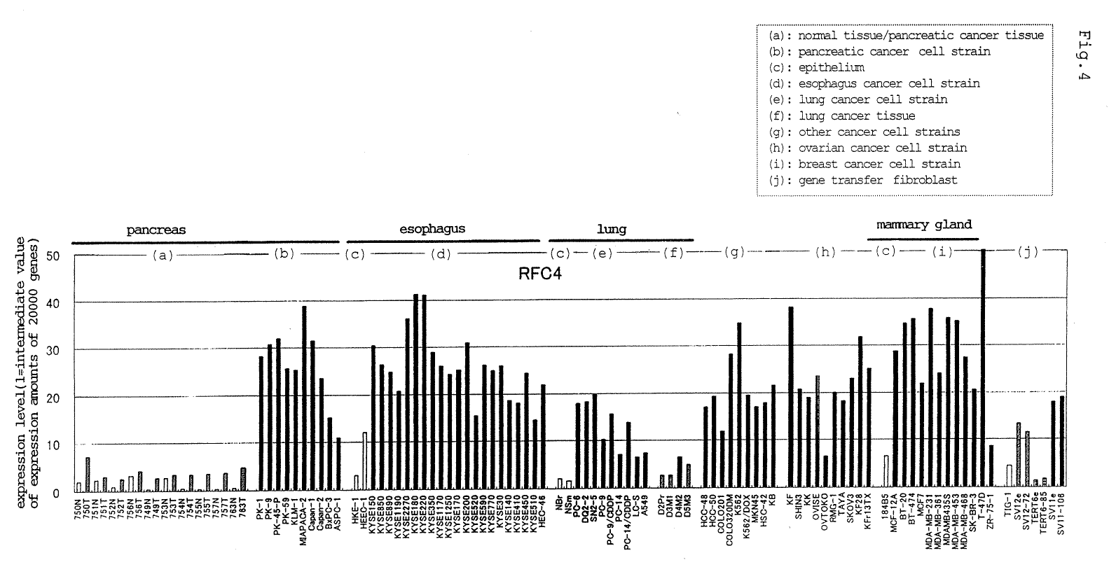

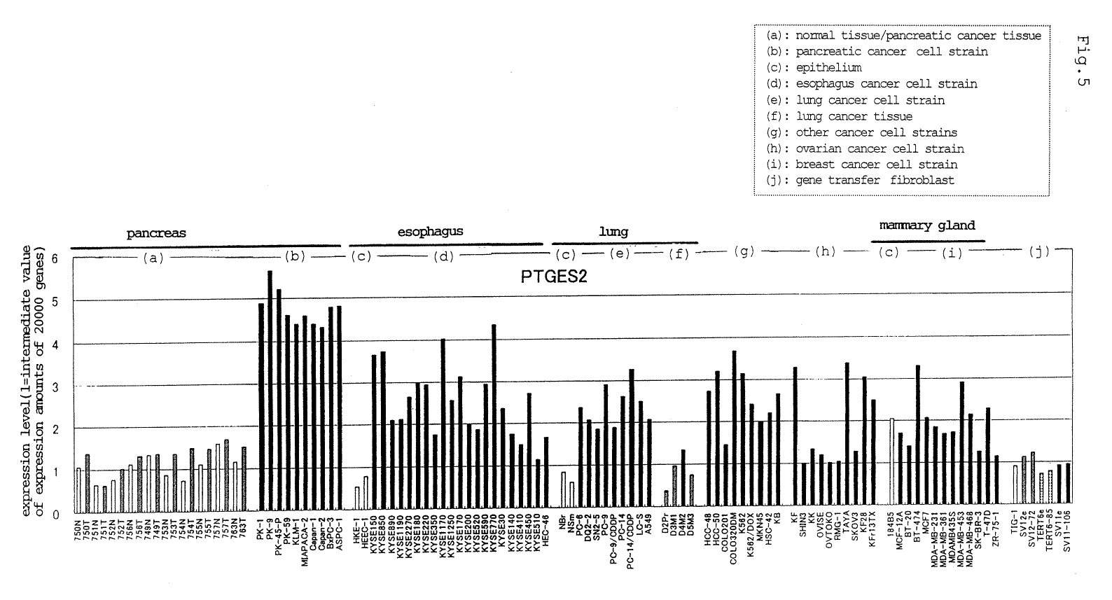

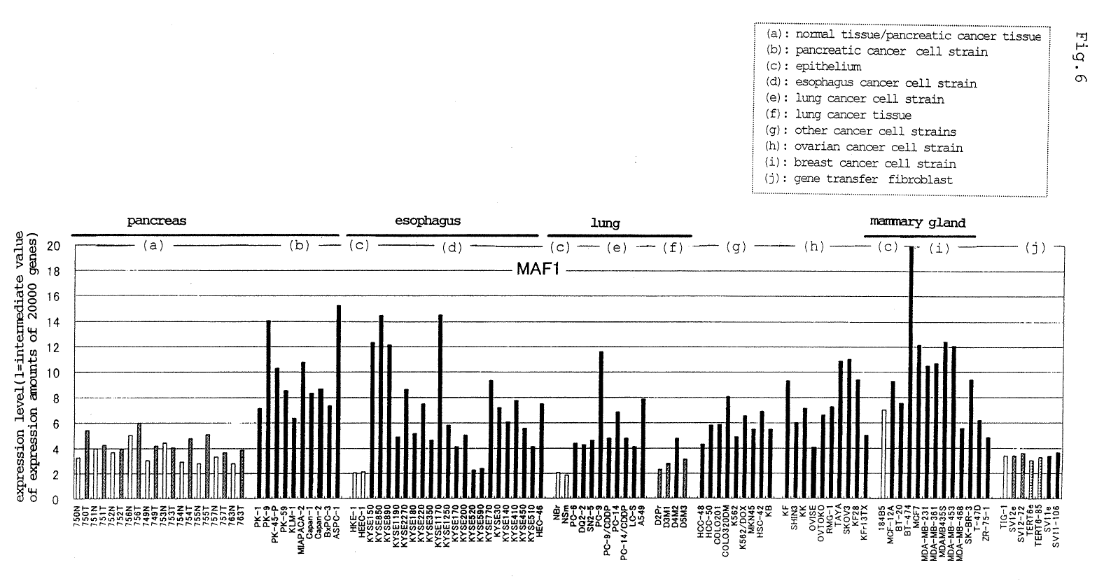

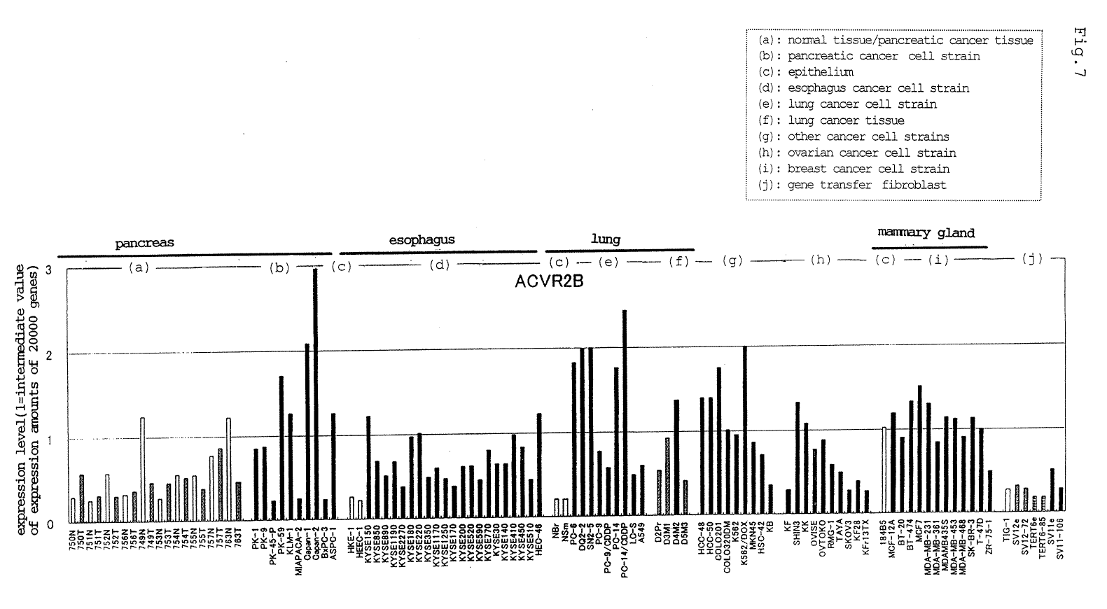

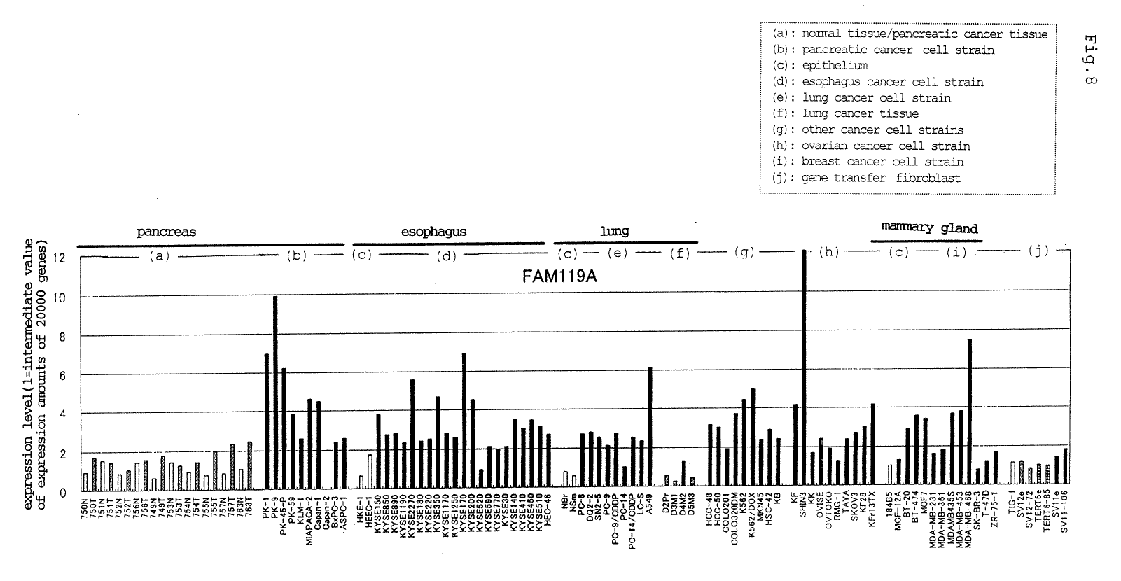

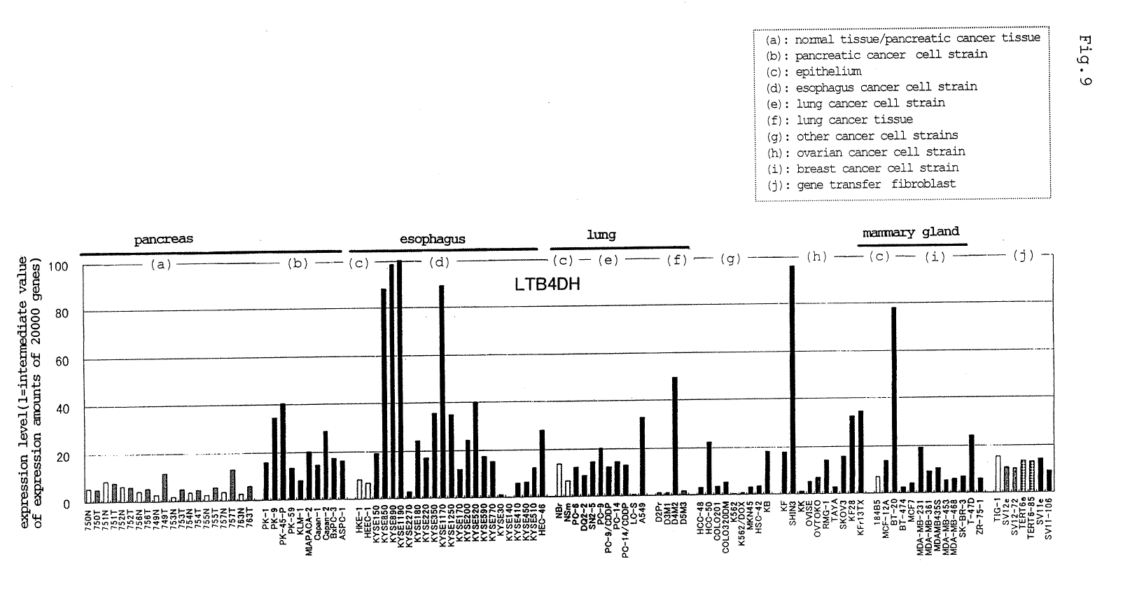

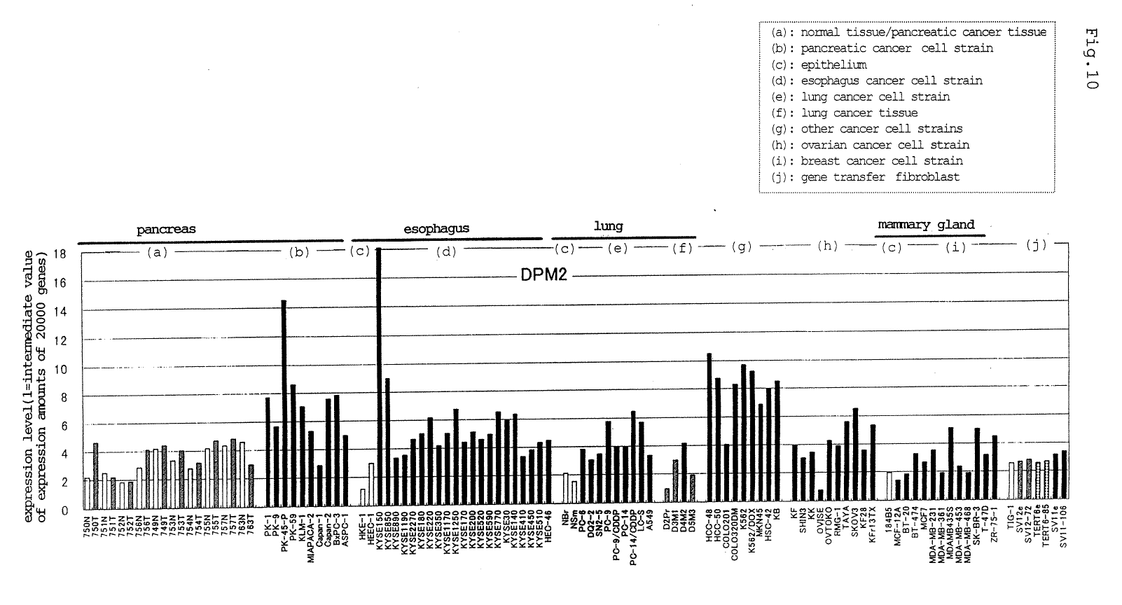

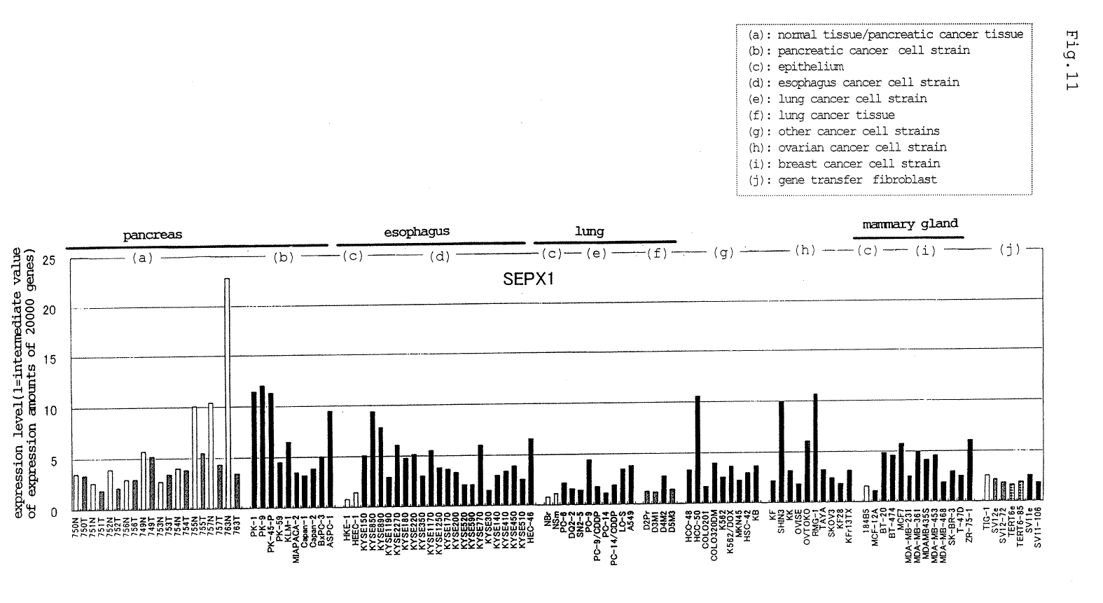

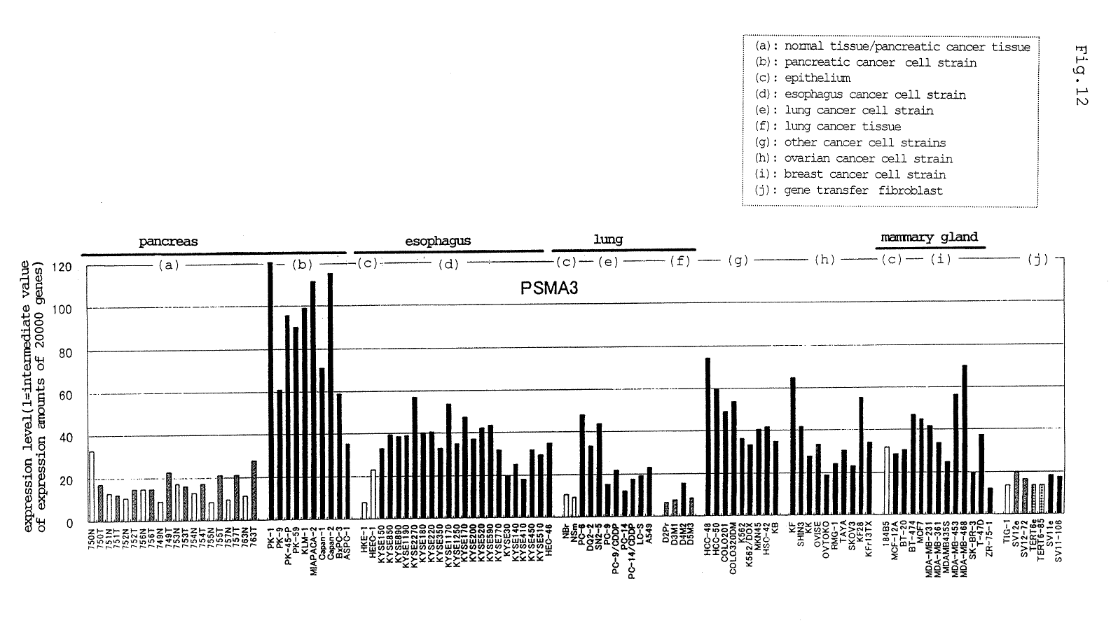

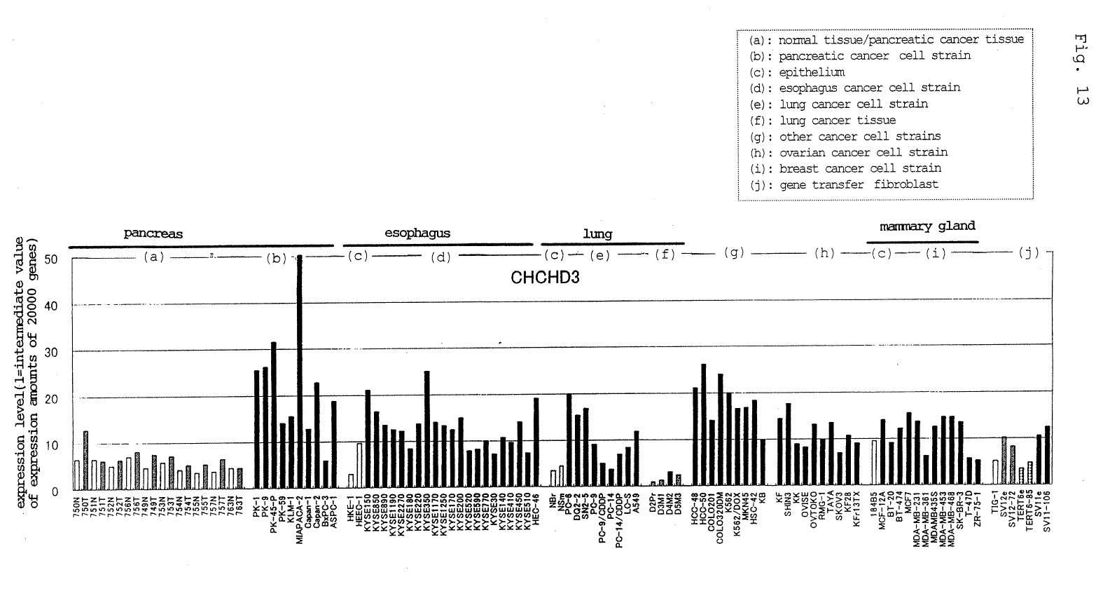

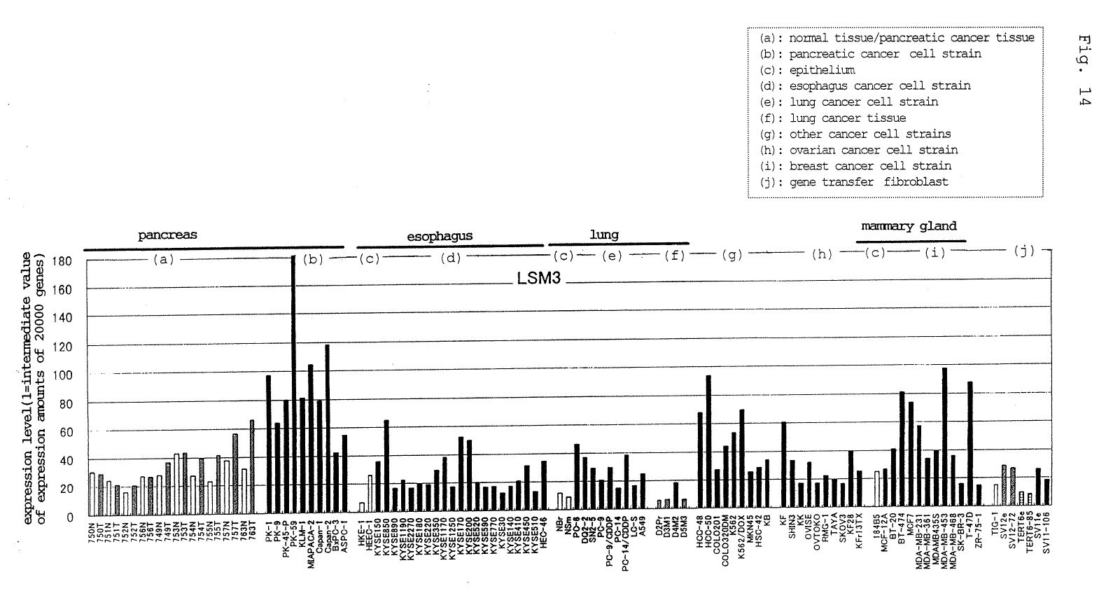

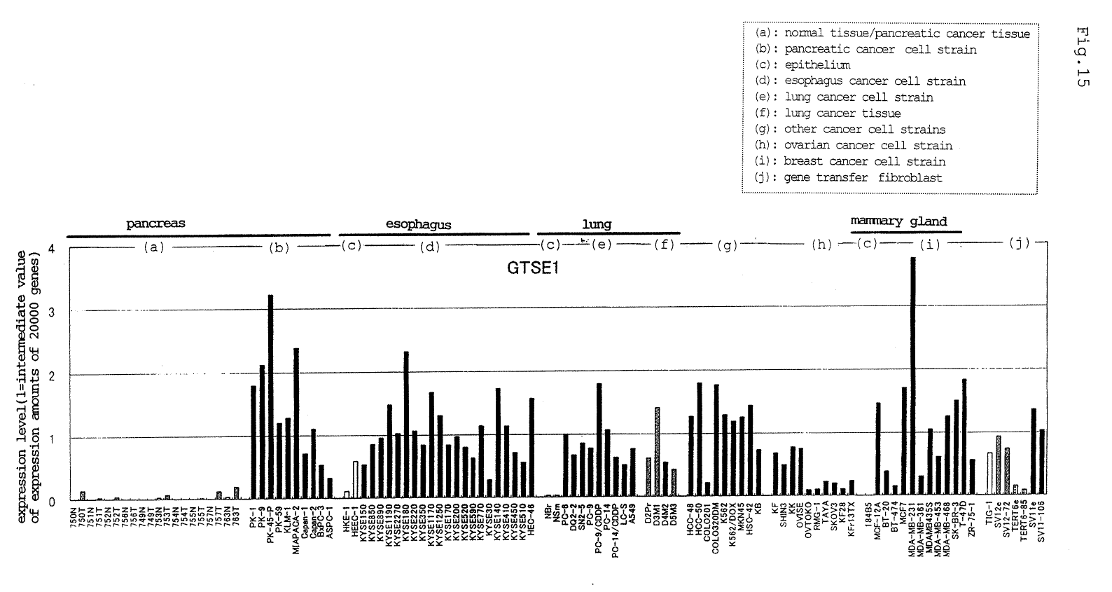

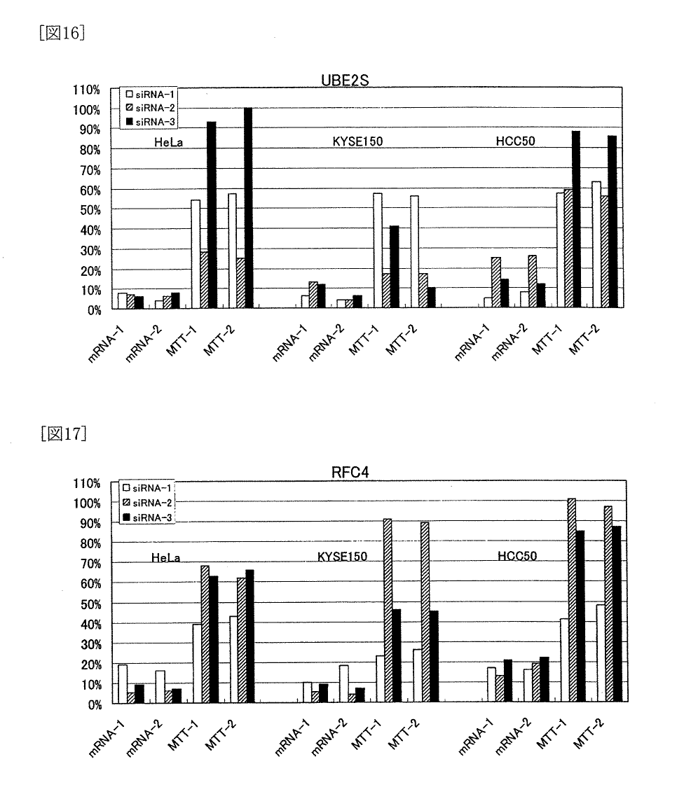

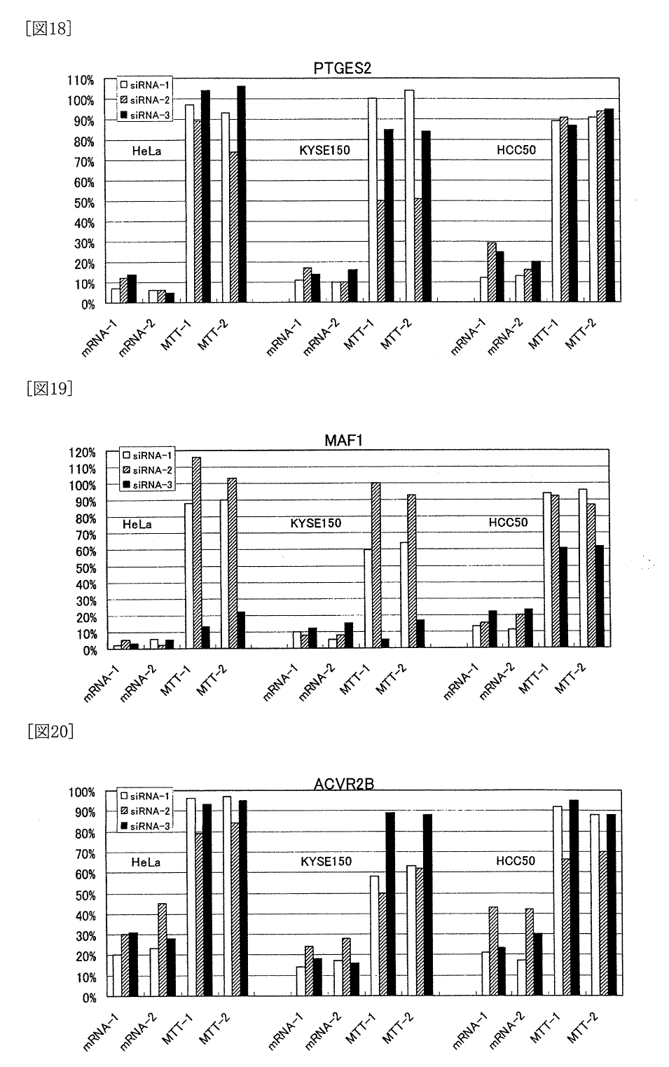

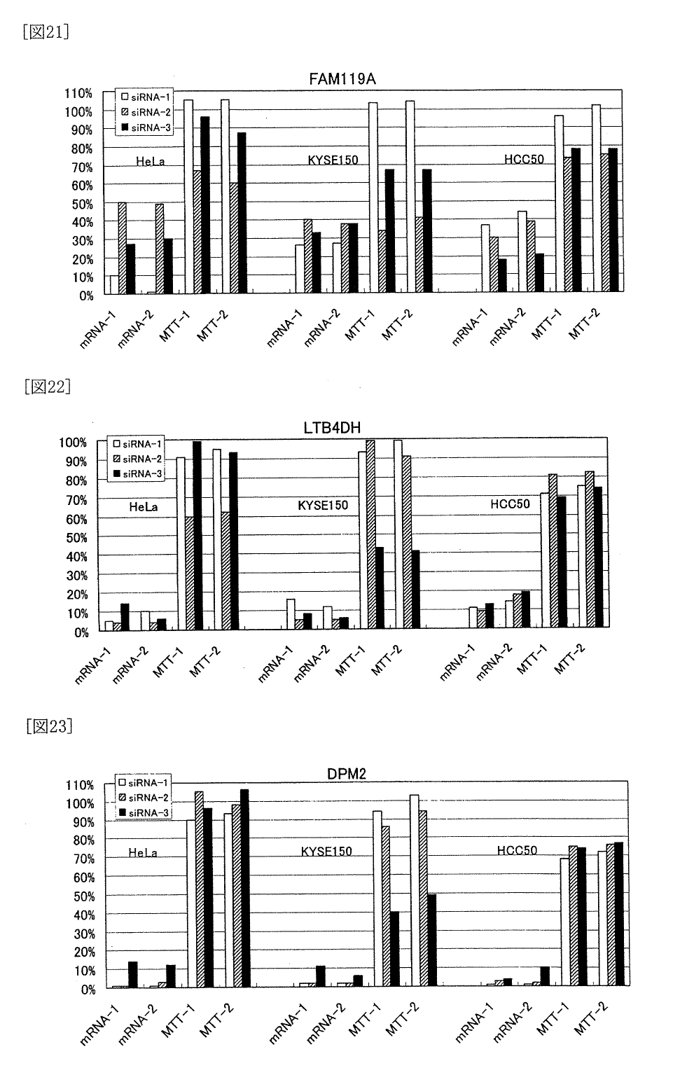

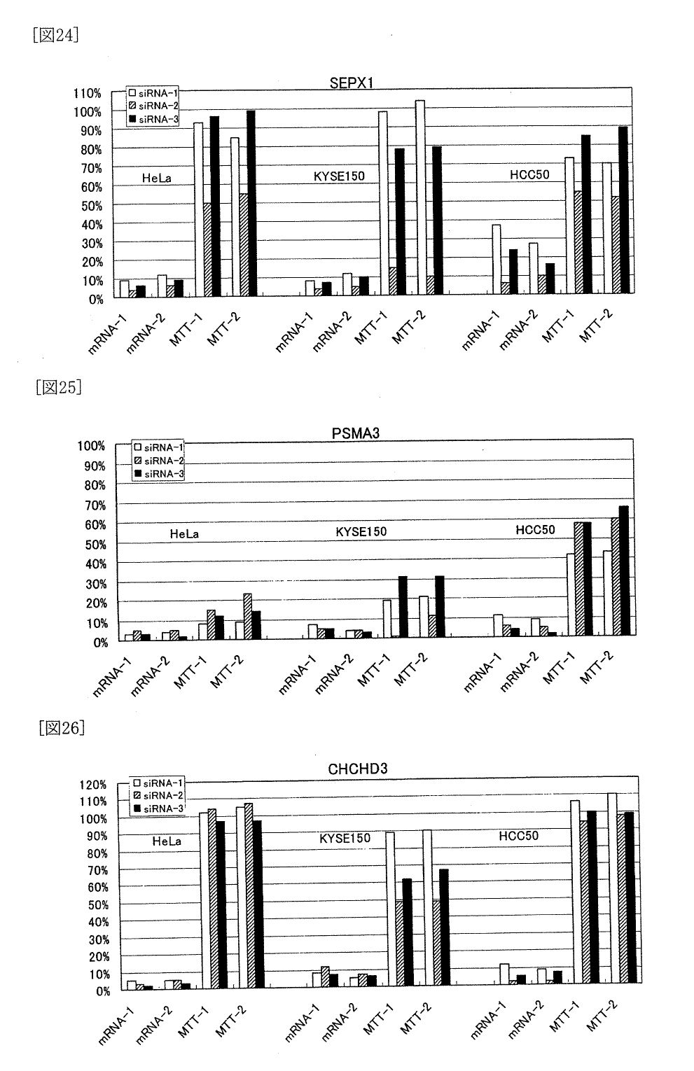

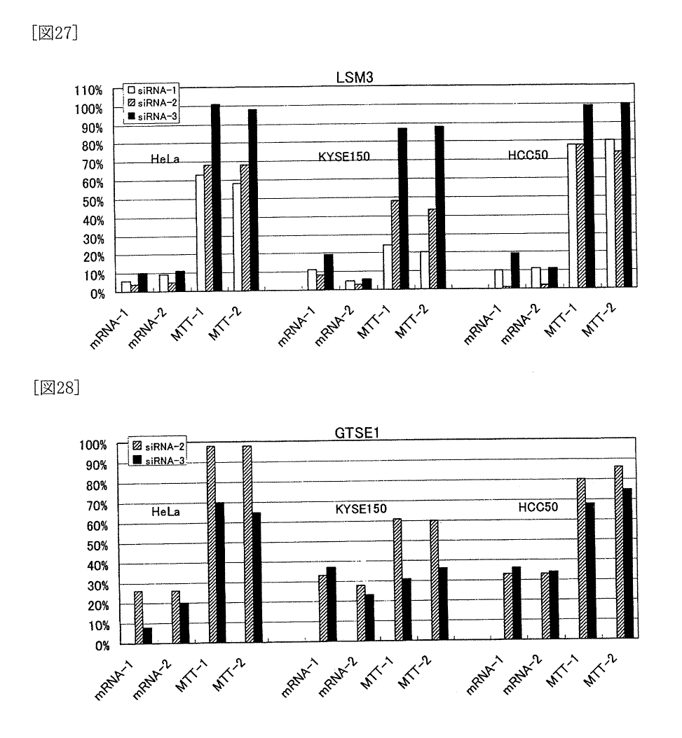

### SEQUENCE LISTING FREE TEXT

SEQ ID No.27 represents the base sequence of the sense strand of siRNA-1 (44-64), of the UBE2S gene.
SEQ ID No.28 represents the base sequence of the sense strand of siRNA-1 (146-166), of the UBE2S gene.
SEQ ID No.29 represents the base sequence of the sense strand of siRNA-2, of the UBE2S gene.
SEQ ID No.30 represents the base sequence of the sense strand of siRNA-3, of the UBE2S gene.
SEQ ID No.31 represents the base sequence of the sense strand of siRNA-1 (897-917), of the RFC4 gene.
SEQ ID No.32 represents the base sequence of the sense strand of siRNA-1 (189-209), of the RFC4 gene.

SEQ ID No.33 represents the base sequence of the sense strand of siRNA-2, of the RFC4 gene.

SEQ ID No.34 represents the base sequence of the sense strand of siRNA-3, of the RFC4 gene.
SEQ ID No.35 represents the base sequence of the sense strand of siRNA-1 (2003-2023), of the PTGES2 gene.
SEQ ID No.36 represents the base sequence of the sense strand of siRNA-1 (1105-1125), of the PTGES2 gene.
SEQ ID No.37 represents the base sequence of the sense strand of siRNA-2, of the PTGES2 gene.

SEQ ID No.38 represents the base sequence of the sense strand of siRNA-3, of the PTGES2 gene.

SEQ ID No.39 represents the base sequence of the sense strand of siRNA-1 (1538-1558), of the MAF1 gene.
SEQ ID No.40 represents the base sequence of the sense strand of siRNA-1 (1031-1051), of the MAF1 gene.
SEQ ID No.41 represents the base sequence of the sense strand of siRNA-2, of MAF1 gene.
SEQ ID No.42 represents the base sequence of the sense strand of siRNA-3, of the MAF1 gene.
SEQ ID No.43 represents the base sequence of the sense strand of siRNA-1 (626-646), of the ACVR2B gene.
SEQ ID No.44 represents the base sequence of the sense strand of siRNA-1 (208-228), of the ACVR2B gene.
SEQ ID No.45 represents the base sequence of the sense strand of siRNA-2, of the ACVR2B gene.
SEQ ID No.46 represents the base sequence of the sense strand of siRNA-3, of the ACVR2B gene.
SEQ ID No.47 represents the base sequence of the sense strand of siRNA-1 (754-774), of the FAM119A gene.
SEQ ID No.48 represents the base sequence of the sense strand of siRNA-1 (1068-1088), of the FAM119A gene.
SEQ ID No.49 represents the base sequence of the sense strand of siRNA-2, of the FAM119A gene.
SEQ ID No.50 represents the base sequence of the sense strand of siRNA-3, of the FAM119A gene.
SEQ ID No.51 represents the base sequence of the sense strand of siRNA-1 (775-795), of the LTB4DH gene.
SEQ ID No.52 represents the base sequence of the sense strand of siRNA-1 (658-678), of the LTB4DH gene.

SEQ ID No.53 represents the base sequence of the sense strand of siRNA-2, of the LTB4DH gene.

SEQ ID No.54 represents the base sequence of the sense strand of siRNA-3, of the LTB4DH gene.
SEQ ID No.55 represents the base sequence of the sense strand of siRNA-1 (145-165), of the DPM2 gene.
SEQ ID No.56 represents the base sequence of the sense strand of siRNA-1 (84-104), of the DPM2 gene.
SEQ ID No.57 represents the base sequence of the sense strand of siRNA-2, of the DPM2 gene.
SEQ ID No.58 represents the base sequence of the sense strand of siRNA-3, of the DPM2 gene.
SEQ ID No.59 represents the base sequence of the sense strand of siRNA-1 (870-890), of the SEPX1 gene.
SEQ ID No.60 represents the base sequence of the sense strand of siRNA-1 (794-814), of the SEPX1 gene.
SEQ ID No.61 represents the base sequence of the sense strand of siRNA-2, of the SEPX1 gene.
SEQ ID No.62 represents the base sequence of the sense strand of siRNA-3, of the SEPX1 gene.
SEQ ID No.63 represents the base sequence of the sense strand of siRNA-1 (853-873), of the PSMA3 gene.
SEQ ID No.64 represents the base sequence of the sense strand of siRNA-1 (686-706), of the PSMA3 gene.
SEQ ID No.65 represents the base sequence of the sense strand of siRNA-2, of the PSMA3 gene.
SEQ ID No.66 represents the base sequence of the sense strand of siRNA-3, of the PSMA3 gene.
SEQ ID No.67 represents the base sequence of the sense strand of siRNA-1 (546-566), of the CHCHD3 gene.
SEQ ID No.68 represents the base sequence of the sense strand of siRNA-1 (1450-1470), of the CHCHD3 gene.
SEQ ID No.69 represents the base sequence of the sense strand of siRNA-2, of the CHCHD3 gene.
SEQ ID No.70 represents the base sequence of the sense strand of siRNA-3, of the CHCHD3 gene.
SEQ ID No.71 represents the base sequence of the sense strand of siRNA-1 (540-560), of the LSM3 gene.
SEQ ID No.72 represents the base sequence of the sense strand of siRNA-1 (37-57), of the LSM3 gene.
SEQ ID No.73 represents the base sequence of the sense strand of siRNA-2, of the LSM3 gene.
SEQ ID No.74 represents the base sequence of the sense strand of siRNA-3, of the LSM3 gene.
SEQ ID No.75 represents the base sequence of the sense strand of siRNA-2, of the GTSE1 gene.
SEQ ID No.76 represents the base sequence of the sense strand of siRNA-3, of the GTSE1 gene.

1. A marker gene for determining cancer cell immortalization, the marker gene comprising a polynucleotide having a base sequence of at least 15 bases that specifically hybridizes with a continuous base sequence of at least 15 bases within any one of base sequences represented by SEQ ID Nos.1 to 13.

2. The marker gene according to claim 1, wherein the marker gene is a probe or primer.

3. A method for determining an immortalized cancer cell, comprising the steps of:
(1) bonding the marker gene according to claim 1 or 2 with RNA prepared from a biological sample that is extracted from a test subject and that may contain a cancer cell, or a derivative of the RNA;
(2) measuring an amount of the RNA or the derivative thereof bonded with the marker gene, using the marker gene as an index; and
(3) comparing the amount of RNA or the derivative thereof found in Step (2) (generically referred to as “an RNA amount”, hereinafter) with an amount (generically referred to as “a comparative RNA amount”, hereinafter) of a corresponding RNA or a derivative thereof in a nonimmortalized healthy or cancer cell.

4. The method for determining an immortalized cancer cell according to claim 3, further comprising the step of:
(4) determining that the cancer cell of the test subject is immortalized when the RNA amount found in Step (2) is higher than the comparative RNA amount, and determining that the cancer cell of the test subject is not immortalized when the RNA amount found in Step (2) is not higher than the comparative RNA amount.

5. An antibody for recognizing a polypeptide having any one of amino acid sequences represented by SEQ ID Nos.14 to 26.

6. A method for determining an immortalized cancer cell, comprising the step of:
(1′) bonding a protein-containing fraction containing a polypeptide prepared from a biological sample that is extracted from a test subject and that may contain a cancer cell with the antibody according to claim 5;
(2′) measuring the amount of the polypeptide that is bonded with the antibody, using the antibody as an index; and
(3′) comparing the amount (generically referred to as “a polypeptide amount”, hereinafter) of the polypeptide found in Step (2′) with the amount (generically referred to as “a comparative polypeptide amount”, hereinafter) of a corresponding polypeptide in a non-immortalized healthy or cancer cell.

7. The method for determining an immortalized cancer cell according to claim 6, further comprising the step of:
(4′) determining that the cancer cell of the test subject is immortalized when the polypeptide amount found in Step (2′) is higher than the comparative polypeptide amount, and determining that the cancer cell of the test subject is not immortalized when the polypeptide amount found in Step (2′) is not higher than the comparative polypeptide amount.

8. An immortalized cancer cell determining reagent kit including at least one member selected from the group consisting of the marker gene according to claim 1 and the antibody according to claim 5.

9. A method for screening a substance for suppressing proliferation of immortalized cancer cells, comprising the steps of:
(A) bringing a test material into contact with a cell that can express one of the immortalization-determining genes represented by SEQ ID Nos. 1 to 13;
(B) measuring an expression amount of one of the immortalization-determining genes represented by SEQ ID Nos. 1 to 13 in the cell brought into contact with the test material;
(C) comparing the expression amount of the immortalization-determining gene found in Step (B) with an expression amount (generically referred to as “a comparative expression amount”, hereinafter) of an immortalization-determining gene in a cell not in contact with the test material; and
(D) selecting the test material as a candidate substance for suppressing proliferation of immortalized cancer cells when the expression amount found in Step (B) is lower than the comparative expression amount.

10. A method for screening a substance for suppressing proliferation of immortalized cancer cells, comprising the steps of:
(A′) bringing a test material into contact with a polypeptide having one of the amino acid sequences represented by SEQ ID Nos. 14 to 26;
(B′) measuring activity of the polypeptide having one of the amino acid sequences represented by SEQ ID Nos. 14 to 26 brought into contact with the test material;
(C′) determining the degree of the activity of the polypeptide found in Step (B′) by comparing the activity of the polypeptide found in Step (B′) with activity (generically referred to as “a comparative activity”, hereinafter) of a polypeptide not in contact with the test material; and
(D′) selecting the test material as a candidate substance for suppressing proliferation of immortalized cancer cells when the activity of the polypeptide found in Step (B′) is lower than the comparative activity.

11. An immortalized cancer cell growth inhibitor containing, as an active ingredient, a polynucleotide having a base sequence of at least 15 bases that specifically hybridizes with a continuous base sequence of at least 15 bases within any one of base sequences represented by SEQ ID Nos. 1 to 13.

12. The immortalized cancer cell growth inhibitor according to claim 11, wherein the polynucleotide has any one of base sequences represented by SEQ ID Nos. 27 to 76.

13. A method of anticancer treatment comprising the step of administering the immortalized cancer cell growth inhibitor according to claim 11 or 12 to cancer cells of a patient.

14. Use of a polynucleotide having a base sequence of at least 15 bases that specifically hybridizes with a continuous base sequence of at least 15 bases within any one of base sequences represented by SEQ ID Nos. 1 to 13, for producing an immortalized cancer cell growth inhibitor.

15. The use according to claim 14, wherein the polynucleotide has any one of base sequences represented by SEQ ID Nos. 27 to 76.
